(19) United States
(12) Patent Application Publication
(10) Pub. No.: US 2011/0106000 A1
(43) Pub. Date: May 5, 2011

(54) Nitric Oxide Compositions and Devices and Methods for Cosmesis

(75) Inventors: Mitchell Lawrence Jones, Montreal (CA); Satya Prakash, Brossard (CA)

(73) Assignee: MICROPHARMA LIMITED, Montreal, QC (CA)

(21) Appl. No.: 13/000,527

(22) PCT Filed: Jun. 23, 2009
(86) PCT No.: PCT/CA2009/000859

§ 371 Date: Dec. 21, 2010

Related U.S. Application Data
(60) Provisional application No. 61/075,040, filed on Jun. 24, 2008.
(60) Provisional application No. 61/097,978, filed on Sep. 18, 2008.
(60) Provisional application No. 61/153,696, filed on Feb. 19, 2009.
(60) Provisional application No. 61/166,430, filed on Apr. 3, 2009.

(51) Int. Cl. A61M 37/00; A61K 8/02; A61K 8/96; A61K 8/66; A61K 8/14; A61K 8/11; A61K 8/99; A61Q 19/08; A61Q 19/00

(52) U.S. Cl. 604/23; 424/401; 424/93.1; 424/94.1; 424/93.45; 424/93.44; 424/93.4; 424/94.6; 424/94.63; 424/94.64; 424/94.5; 424/94.4

(57) ABSTRACT

The present disclosure provides a device having a casing with a barrier surface and a contact surface and a composition in the casing having a nitric oxide precursor and an isolated enzyme or live cell expressing an endogenous enzyme, for converting the nitric oxide gas precursor to nitric oxide gas or having activity on a substrate that produces a catalyst that causes the conversion of the nitric oxide gas precursor to nitric oxide gas. The present disclosure also provides compositions, methods and uses for skin cosmesis.

### FIELD OF THE DISCLOSURE

The present disclosure relates to devices, compositions and methods for skin cosmesis with nitric oxide.

### BACKGROUND OF THE DISCLOSURE

While there is a long history of topically applied chemicals and botanicals, some attached to specific health benefits, there has only recently been a significant research effort to define the biologically active compounds and to elucidate the mechanisms of action (Syed et al. 2007; Vayalil et al. 2007). Many commercially available products have relied on consumers' somewhat limited understanding of human physiology, cell biology, and new scientific discovery, purporting to use complex enzymes and other bioactives for cosmesis, such as reducing the effects of aging.

The dichotomy is that there are many organically derived materials that contain some bioactive ingredients and that may have some biologic effect; however, most commercially available products either contain too little bioactive or simply purport science that is not true. A common difficulty is that the active compound is often only naturally available at lower concentrations than its minimal therapeutic concentration (Liu and Wang, 2007). Also, some bioactives, such as anti-oxidants, are required at therapeutic levels for long durations to achieve a maximal therapeutic effect. Thus, while organically derived skin care products may be active, or chemically synthesised bioactives may be added, they may not be achieving a therapeutic effect because of low concentration or limited duration of action.

While nitric oxide has many beneficial effects in pathological states, so too it may benefit normal skin cosmesis. Normally, NO is synthesized in small amounts by mammalian cells from L-arginine by endothelial nitric oxide synthase (NOS) for cell signalling. Nitric oxide is generated in much larger quantities by skin and inflammatory cells during inflammatory reactions by the inducible NOS. Also, microorganisms found on skin and in dermis have nitrate reductase (NiR) activity and produce gNO from nitrate in sweat and saliva (Benjamin et al. 1997; Weller et al. 1996). Nitrate present in the blood, and later in sweat, is acted on by the NiR possessed by these microorganisms (Benjamin et al. 1997; Weller et al. 1996). Superficial antimicrobial agents (chlorhexadine and other topical antibiotics) cannot kill these organisms and cannot diminish the gNO produced by skin. If you treat locally with a nitric oxide synthase (NOS) inhibitor (L-NMMA), you do not decrease the amount of NO produced and released by the skin (Hardwick et al. 2001). However, if you treat with systemic wide-spectrum antibiotics, for an adequate duration, the skin loses it's NO producing capability (Weller et al. 1996). Interestingly, the quantity of NO produced by the hands is much greater than that produced by the arm—this makes sense as the hands have a much greater need for the antimicrobial effects of NO (Weller et al. 1996). Thus, there is a “map” of NO producing capability of the skin which is a product of the density of sweat glands and the presence/absence of bacteria with nitrate reductase (NiR) activity.

The appearance of skin can be enhanced by improved extracellular matrix deposition (ECM), increased moisture content, control of keratinocyte division and migration, improved blood flow, reduced inflammation, reduced pathogenic load, and increased anti-oxidative capacity. Nitric oxide can affect many of these factors and may have particular relevance by increasing regional blood flow and encouraging improved orderly collagen deposition.

### SUMMARY OF THE DISCLOSURE

The present inventors have developed a composition and device in which free enzyme or bacteria, supported by compositional growth media and body temperature, can act on substrate for the continuous production of therapeutically relevant nitric oxide gas (gNO). The composition is typically a time-release composition. Compositions and devices containing bacteria or enzyme isolates that act on substrate to produce gNO will be effective in cosmesis and have considerable commercial value.

In particular, the inventors have shown that microorganisms can be used for sustained production of controlled amounts of nitric oxide (NO). Biosynthesis of NO through the denitrification pathway from nitrate is a well known mechanism in microorganisms and this application provides the first disclosure of methods of skin cosmesis using such gas. Some lactobacilli reduce nitrate (NO₃⁻) to nitrite (NO₂⁻) and NO under anaerobic conditions (nitrate reductase) (Wolf et al. 1990). Other microorganisms produce NO by metabolism of L-arginine (NOS enzyme) nitrate in the growth medium under anaerobic conditions (Xu and Verstraete, 2001).

Immobilized bacteria or free enzyme, in the presence of precursor substrates, can produce NO over the desired therapeutic time and at therapeutically relevant levels. The therapeutic capability of the bacteria or enzyme is maintained over the period of time in which they have sufficient nutrients, are not surrounded by excess waste, and have the substrate and cofactors required to be biochemically efficient at producing the therapeutic gas.

Accordingly, the present application discloses methods, compositions and devices for cosmesis of skin using a topical source of nitric oxide.

In an aspect, the application provides a composition for delivering nitric oxide gas topically to skin. In an embodiment, the application provides a composition for delivering nitric oxide gas to skin comprising (a) an isolated enzyme or a live cell expressing an endogenous enzyme, the enzyme (i) having activity that converts a nitric oxide gas precursor to nitric oxide gas or (ii) having activity on a substrate that produces a catalyst that causes the conversion of the nitric oxide gas precursor to nitric oxide gas, or (b) a live cell producing a catalyst for converting a nitric oxide gas precursor to nitric oxide gas; and a carrier. In an embodiment, the nitric oxide gas precursor is present on the skin of the subject, for example, in the form of nitrate produced from sweat. In another embodiment, the composition further comprises a nitric oxide gas precursor. In yet another embodiment, the carrier comprises a matrix.

In another aspect, the application provides a device for delivering nitric oxide gas topically to skin comprising the compositions described herein. In one embodiment, the application provides a device for delivering nitric oxide gas to skin comprising a casing having a barrier surface and a contact surface that is permeable to nitric oxide gas; and a composition in the casing that is comprised of i) a nitric oxide gas precursor, and ii) (a) an isolated enzyme or a live cell expressing an endogenous enzyme, the enzyme 1) having activity that converts the nitric oxide gas precursor to nitric oxide gas or 2) having activity on a substrate that produces a catalyst that causes the conversion of the nitric oxide gas precursor to nitric oxide gas, or (b) a live cell producing a catalyst for converting the nitric oxide gas precursor to nitric oxide gas.

In another embodiment, the device further comprises a nitric oxide gas concentrating agent.

In yet another embodiment, the casing comprises a plurality of layers. In one embodiment, the layers include a barrier layer; a contact layer; and an active layer. In another embodiment, the active layer comprises the composition; the barrier layer comprises the barrier surface and the contact layer comprises the contact surface. In a further embodiment, the casing also includes a reservoir layer. In one embodiment, the reservoir layer comprises the nitric oxide gas precursor. In yet another embodiment, the casing also includes a trap layer. In one embodiment, the trap layer comprises the nitric oxide gas concentrating agent.

In another aspect, the application provides methods and uses of a device or composition of the application for cosmesis of skin in a subject in need thereof.

In one aspect, the application provides a method for skin cosmesis in a subject in need thereof comprising

contacting the skin with a casing permeable to nitric oxide gas, the casing containing a plurality of inactive agents that, upon activation, react to produce nitric oxide gas;

activating the inactive agents to produce nitric oxide gas, wherein the nitric oxide gas communicates through the casing and contacts the skin for cosmesis in the subject in need thereof.

In another aspect, the application provides a method for skin cosmesis in a subject in need thereof comprising

contacting the skin with a nitric oxide gas releasing composition, the composition containing a plurality of inactive agents that, upon activation, react to produce nitric oxide gas;

activating the inactive agents to produce nitric oxide gas, wherein the nitric oxide gas contacts the skin for cosmesis in the subject in need thereof.

In an embodiment, the inactive agents are separated and activation of the inactive agents comprises combining the separated agents together by mixing the separated agents only after an applied pressure or temperature. In another embodiment, the inactive agents are dehydrated agents and activation of the inactive agents comprises hydration.

In another embodiment, the inactive agents comprise i) a nitric oxide gas precursor, and ii) (a) an isolated enzyme or a live cell expressing an endogenous enzyme, the enzyme (i) having activity that converts the nitric oxide gas precursor to nitric oxide gas or (ii) having activity on a substrate that produces a catalyst that causes the conversion of the nitric oxide gas precursor to nitric oxide gas or (b) a live cell producing a catalyst for converting the nitric oxide gas precursor to nitric oxide gas.

In yet another aspect, the disclosure provides a method for cosmesis of skin in a subject in need thereof comprising exposing the skin to a device or composition of the application, wherein NO produced by the device or composition contacts the skin for a treatment period without inducing toxicity to the subject or healthy tissue. The treatment period will depend on the type of device or composition used. For example, for a device described herein, the treatment period typically is from about 1 to 24 hours, preferably about 6-10 hours and more preferably about 8 hours. For a composition contained in a patch, the treatment period typically is from about 1 to 8 hours. For a cream composition, the cream is typically applied one to three times daily. For a mask composition, the treatment period is typically from about 1 to 8 hours, optionally, 1-2 hours.

In yet a further embodiment, the NO is produced by the device or composition from about 3 to 160 parts per million volume (ppmv), preferably from about 25 to 125 ppmv.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure will now be described in relation to the drawings in which:

FIG. 1 shows the concentration of nitric oxide gas (gNO) released by MRS agar growing Lactobacillus fermentum (ATCC 11976) supplemented with several concentrations of NaNO₂. The concentration of gNO produced by MRS medium growing Lactobacillus fermentum (ATCC 11976) supplemented with a 40 cm² Nitro-Dur 0.8 mg/hr nitro-glycerine transdermal patch (GTN) (Key Pharmaceuticals) is also shown. Measurements were made after 20 hours of growth at 37° C. without shaking.

FIG. 2 shows nitric oxide gas (gNO) released by medium growing Lactobacillus fermentum (ATCC 11976) with the indicated concentrations of NaNO₂ or Escherichia coli BL21 (pnNOS) (pGroESL) with the indicated cofactors. Measurements were made after 20 hours of growth at 37° C. without shaking.

FIG. 3A shows nitric oxide gas released by the medium growing either Lactobacillus plantarum LP80, Lactobacillus fermentum (ATCC 11976), Lactobacillus ferment (NCIMB 2797) or Lactobacillus fermentum (LMG 18251) with the indicated concentrations of KNO₃ or NaNO₂. Measurements were made after 20 hours of growth at 37° C. without shaking. FIG. 3B shows nitrite released by the medium growing either Lactobacillus plantarum LP80, Lactobacillus fermentum (ATCC 11976), Lactobacillus fermentum (NCIMB 2797) or Lactobacillus fermentum (LMG 18251) with the indicated concentrations of KNO₃ or NaNO₂. Measurements were made after 20 hours of growth at 37° C. without shaking. FIG. 3C shows nitrate released by the medium growing either Lactobacillus plantarum LP80, Lactobacillus fermentum (ATCC 11976), Lactobacillus fermentum (NCIMB 2797) or Lactobacillus fermentum (LMG 18251) with the indicated concentrations of KNO₃ or NaNO₂. Measurements were made after 20 hours of growth at 37° C. without shaking.

FIG. 4 is a series of graphs that show that lactic acid bacteria produce low pH in media and that produced protons act on nitrite to produce nitric oxide. FIG. 4A is a graph that shows the pH of the medium growing Lactobacillus fermentum (ATCC 11976) with the indicated concentrations of NaNO₂ and 20 g/L (no glucose added) or 100 g/L (glucose added) glucose. Measurements were made after the indicated number of hours at 37° C. without shaking. FIG. 4B is a graph that shows the optical density of the medium growing Lactobacillus fermentum (ATCC 11976) with the indicated concentrations of NaNO₂ and 20 g/L (no glucose added) or 100 g/L (glucose added) glucose. Measurements were made after 3, 4, 5, 6, and 20 hours at 37° C. without shaking. FIG. 4C is a nitric oxide gas released by the medium growing Lactobacillus fermentum (ATCC 11976) with the indicated concentrations of NaNO₂ and 20 g/L (no glucose added) or 100 g/L (glucose added) glucose. Measurements were made after the indicated number of hours at 37° C. without shaking.

FIG. 5 shows a graphical representation of the relative quantity of nitric oxide gas (gNO), as represented by area under the curve, produced by strains of Lactobacillus fermentum grown in MRS media at 37° C. for 20 hours.

FIG. 6 shows a repeat of the relative quantity of nitric oxide gas (gNO), as represented by area under the curve, produced by strains of Lactobacillus fermentum grown in MRS media at 37° C. for 20 hours.

FIG. 7 shows the head gas pressure (kPa) in the vessel where strains of Lactobacillus fermentum were grown in MRS media at 37° C. for 20 hours.

FIG. 8 shows nitrate (NO₃) produced by strains of Lactobacillus fermentum grown in MRS media at 37° C. for 20 hours.

FIG. 9 shows nitrite (NO₂) produced by strains of Lactobacillus fermentum grown in MRS media at 37° C. for 20 hours.

FIG. 10 shows nitric oxide gas produced by Lactobacillus reuteri (NCIMB 701359), Lactobacillus reuteri (LabMet) and Lactobacillus fermentum (ATCC 11976) in the presence of ½ patch of nitroglycerin (first 4 columns) or in the presence of ½ patch of nitroglycerin with the addition of P450 or gluthathione-S-transferase inhibitors (last 3 columns).

FIG. 11 shows a multilayered nitric oxide producing device.

FIG. 12 shows a simple single layered device.

FIG. 13 shows another simple layered device.

FIG. 14 shows yet another simple layered device.

FIG. 15 shows a schematic of a composition of probiotic bacteria (NiR+FAE active L. fermentum) and glucose substrate.

FIG. 16 shows gaseous nitric oxide gNO produced by probiotic lotion.

FIG. 17 shows gaseous nitric oxide (gNO) produced by a two component antioxidant skin cream containing probiotic bacteria.

FIG. 18 shows contribution of individual components of probiotic, antioxidant containing face cream to nitric oxide production.

FIG. 19 shows pH dependence of gaseous nitric oxide (gNO) production by ascorbic-6-phosphate containing skin cream.

FIG. 20 shows production of nitric oxide by a simple gel containing probiotic bacteria.

FIG. 21 shows nitric oxide production by alginate skin mask containing L. fermentum NCIMB 7230.

FIG. 22 shows in vivo nitric oxide production by alginate skin mask containing L. fermentum NCIMB 7230.

FIG. 23 shows blood flow response to treatment with nitric oxide producing alginate based skin mask.

FIG. 24 shows long term changes in blood flow caused by treatment with L. fermentum NCIMB 7230 skin mask.

FIG. 25 shows the effect of sodium nitrite concentration on nitric oxide production by L. fermentum NCIMB 7230 nitric oxide face mask.

FIG. 26 shows the enzymatic gaseous nitric oxide (gNO) production with 1% bromelain, varying gelatin at room temperature and at 37° C.

FIG. 27 shows enzymatic production of gaseous nitric oxide (gNO) with 10% gelatin, varying bromelain at room temperature and at 37° C.

FIG. 28 shows enzymatic production of gaseous nitric oxide (gNO) with 20% bromelain, varying gelatin at room temperature and at 37° C.

FIG. 29 shows enzymatic generation of gaseous nitric oxide (gNO) by patches containing 10% bromelain.

FIG. 30 shows enzymatic production of gaseous nitric oxide (gNO) with 0.1% bromelain and M-N-A-Benzoyl-L-arginine ethyl ester (BAEE) at 30° C.

FIG. 31 shows the generation of gNO measured hourly in the presence of porcine liver esterase, sodium nitrite, and various ester substrates. A minimum target production was achieved one hour after path activation. No gNO was detected using controls in which neither substrate (triacetin) nor enzyme were present. The best substrates for porcine liver esterase are triacetin and ethyl acetate.

FIG. 32 shows the generation of gNO measured hourly in the presence of candida rugosa lipase (“CRL”), sodium nitrite, and various ester substrates. A minimum target gNO production of 200 ppmV was achieved with triacetin as a substrate, one hour after the reaction was started.

FIG. 33 shows the generation of gNO measured hourly in the presence of triacetin, sodium nitrite, and various enzymes. No gNO production was obtained in the absence of substrate or enzyme. Candida rugosa lipase and porcine liver esterase are the best enzymes for triacetin.

FIG. 34 shows the generation of gNO analyzed in the presence of sodium nitrite, porcine liver esterase and varying concentrations of triacetin. No gNO production was observed in the absence of enzyme or substrate (triacetin).

FIG. 35 shows the generation of gNO evaluated hourly in 4 patches containing triacetin, CRL, alginate microbeads and sodium nitrate. A target production gNO of over 200 ppmV was reached 2 hours after patch activation and it was sustained up to 30 hours.

### DETAILED DESCRIPTION OF THE DISCLOSURE

The present application provides a topical device and a topical composition for administration of nitric oxide to skin capable of continually producing nitric oxide production and its methods and uses.

### Compositions and Devices

In one aspect, the disclosure provides a topical composition comprising (a) an isolated enzyme or a live cell expressing an endogenous enzyme, the enzyme (i) having activity that converts the nitric oxide gas precursor to nitric oxide gas or (ii) having activity on a substrate that produces a catalyst that causes the conversion of the nitric oxide gas precursor to nitric oxide gas, or (b) a live cell producing a catalyst for converting the nitric oxide gas precursor to nitric oxide gas; and a carrier. In one embodiment, the nitric oxide gas precursor is present on the skin, for example, from nitrate produced in sweat. In another embodiment, the composition further comprises a nitric oxide gas precursor.

The term “topical composition” as used herein refers to any substance that comprises the enzyme, live cell or catalyst and optionally, the nitric oxide precursor, and can be applied directly to skin. In one embodiment, the topical composition is a cream, slab, gel, hydrogel, dissolvable film, spray, paste, emulsion, patch, liposome, balm, powder or mask or combination thereof. In another embodiment the composition is two separate parts.

In an embodiment, a first part comprises a substrate or nitric oxide gas precursor and a second part comprises an enzyme for conversion of the precursor into gNO or a catalyst. In a further embodiment, at least one part further comprises at least one antioxidant or reducing agent. For example where the first and second parts are emulsions, the first part may comprise oils, surfactants, water, and a substrate, such as nitrate and the second part may comprise oils, surfactants, water and an enzyme, such as NiR containing total cell extract or a NiR containing cell membrane extract. The first or second part optionally also comprises at least one antioxidant, optionally dithionite, menaquinone, ubiquinone, vitamin K, vitamin E or vitamin C. The first and second parts are then brought together at the time of application. Alternatively, the nitric oxide gas precursor may be found in the skin of the subject. In such an embodiment, the composition comprises a catalyst or enzyme. In one embodiment, the composition comprises an enzyme, such as NiR containing total cell extract, or a NiR containing cell membrane extract, and optionally an antioxidant, optionally, dithionite, menaquinone, ubiquinone, vitamin K, vitamin E or vitamin C and upon application the enzyme reacts with the nitrate from the skin to generate gNO in situ. In one embodiment, the composition is an emulsion and comprises oil, water and surfactants.

In one embodiment, the carrier comprises a matrix. A person skilled in the art can readily determine a suitable matrix for topical application. The matrix may include, without limitation, a natural polymer, such as alginate, chitosan, gelatin, cellulose, agarose, locust bean gum, pectin, starch, gellan, xanthan and agaropectin; a synthetic polymer, such as polyethyleneglycol (PEG), polyacrylamide, polylacticacid (PLA), thermoactivated polymers and bioadhesive polymers; a gel or hydrogel, such as petroleum jelly, intrasite, and lanolin or water-based gels; hydroxyethylcellulose and ethyleneglycol dglycidylether (EDGE); a dissolvable film polymer such as hydroxymethylcellulose; a microcapsule or liposome; and lipid-based matrices. Intrasite is a colourless transparent aqueous gel, which typically contains a modified carboxymethylcellulose (CMC) polymer together with propylene glycol as a humectant and preservative, optionally 2.3% of a modified carboxymethylcellulose (CMC) polymer together with propylene glycol (20%).

Other matrix components, include, without limitation, vitamin A, vitamin B, vitamin C, vitamin D, vitamin E, vitamin K, zinc oxide, ferulic acid, caffeic acid, glycolic acid, lactic acid, tartaric acid, salicylic acid, stearic acid, sodium bicarbonate, salt, sea salt, aloe vera, hyaluronic acid, glycerine, silica silylate, polysorbate, purified water, witch hazel, coenzyme, soy protein (hydrolysed), hydrolyzed wheat protein, methyl & propyl paraben, allantoin, hydrocarbons, petroleum jelly, rose flower oil (rosa damascens), lavender and other typical moisturizers, softeners, antioxidants, anti-inflammatory agents, vitamins, revitalizing agents, humectants, coloring agents and/or perfumes known in the art.

In an embodiment, the composition is applied to a bandage, dressing or clothing.

In another aspect, the application provides a device comprising the compositions described herein. In one embodiment, the device comprises a casing comprising a barrier surface and a contact surface, said contact surface being permeable to nitric oxide gas, wherein the casing comprises a composition described herein, and the composition is located between the barrier surface and the contact surface. The barrier surface is optionally connected to the contact surface so that the barrier surface and contact surface define a cavity in which the composition is located. Typically the barrier surface is connected to the contact surface proximate to the perimeter of the contact surface so that the barrier surface surrounds the perimeter thereof, thereby requiring NO gas to leave only through the contact surface. In an embodiment, the application provides a device for delivering nitric oxide gas to skin, comprising

a casing comprising a barrier surface and a contact surface, said contact surface being permeable to nitric oxide gas; and

a composition in the casing, the composition comprising i) a nitric oxide gas precursor, and ii) (a) an isolated enzyme or a live cell expressing an endogenous enzyme, the enzyme 1) having activity that converts the nitric oxide gas precursor to nitric oxide gas or 2) having activity on a substrate that produces a catalyst that causes the conversion of the nitric oxide gas precursor to nitric oxide gas, or (b) a live cell producing a catalyst for converting the nitric oxide gas precursor to nitric oxide gas.

In one embodiment, the casing separates the composition from the skin and the casing is impermeable to the composition.

The term “casing” as used herein means a shell that retains the composition, and wholly or partially covers the composition. In one embodiment, the casing is a series or plurality of layer(s), for example, flexible and/or rigid laminate. In another embodiment, the casing is a bag or a container. The term “in the casing” as used herein means wholly or partially covering and retaining the composition such that the composition is separated from tissue.

The term “contact surface” as used herein means the surface of the casing that directly interacts with the skin and can be made of any suitable material such as a non-occlusive dressing.

The term “barrier surface” as used herein means the surface of the casing that is not directly contacting the skin, that is, the entire surface of the casing except for the contact surface which directly contacts the skin. The barrier surface may be permeable or impermeable to oxygen. The barrier surface may be made of any suitable material such as plastic. In another embodiment, the barrier surface comprises an adhesive layer that adheres to the skin. In a particular embodiment, the barrier surface is oxygen permeable, protects the skin and adheres to the skin.

In another embodiment, the layers of the casing comprise a barrier layer, a contact layer and an active layer. In a particular embodiment, the active layer comprises the composition, the barrier layer comprises the barrier surface and the contact layer comprises the contact surface. In another embodiment, the casing further comprises a reservoir layer. In one embodiment, the active layer comprises the cell or enzyme and the reservoir layer comprises the nitric oxide gas precursor.

In a further embodiment of the device, the casing further comprises a trap layer. In one embodiment, the trap layer comprises the nitric oxide gas or radical concentrating substance.

The term “nitric oxide gas” or “gNO” or “NO g” as used herein refers to the chemical compound NO and is also commonly referred to as nitric oxide radical.

The term “enzyme” as used herein is intended to include any enzyme or fragment thereof capable of converting a nitric oxide precursor to nitric oxide gas either directly or through the production of a catalyst that causes the conversion of the nitric oxide gas precursor to nitric oxide gas.

In one embodiment, the enzyme is a glutathione S-transferase (GST) or cytochrome P450 system (P450).

In another embodiment, the enzyme is nitric oxide synthase enzyme (NOS) or nitric oxide reductase (NiR). In an embodiment, the enzyme is all or part of the nitric oxide synthase enzyme having NOS activity. In a particular embodiment, the NOS comprises the amino acid sequence as shown in SEQ ID NO:1 or Table 1. In another embodiment, the enzyme is all or part of the nitric oxide reductase having NIR activity. In a particular embodiment the NiR comprises several subunits with amino acid sequences as shown in SEQ ID NOs:2-5 or Table 1. The enzyme optionally is contained in a protein fraction isolated from cells.

The term “catalyst” or “nitric oxide gas precursor reducing agent” as used herein means a substance that causes the conversion of the nitric oxide gas precursor to nitric oxide gas and can be through a dismutation reaction. Further, the catalyst may be produced through the reaction of an enzyme with a substrate. In another embodiment, the catalyst is lactic acid, acetic acid, sulfuric acid, hydrochloric acid or other weaker organic acids. In a particular embodiment, the catalyst is lactic acid. In another embodiment, the catalyst comprises protons. In one embodiment, the protons are a product of the reaction of the enzyme with the substrate. The term “product of the reaction” as used herein includes both products and/or by-products of the enzyme reaction.

In one embodiment, the catalyst producing enzyme is from a bromelain solution, an extract from pineapple. Bromelain as used herein refers to a crude, aqueous extract from the stems and immature fruits of pineaples (Ananas comosus Merr., mainly var. Cayenne from the family of bromeliaceae), constituting an unusually complex mixture of different thiol-endopeptidases and other not yet completely characterized components such as phosphatases, glucosidases, peroxidases, cellulases, glycoproteins and carbohydrates, among others. In addition, bromelain contains several proteinases inhibitors. In one embodiment, the enzyme and substrate that produce a catalyst comprises bromelain, which contains both enzyme and substrate, bromelain and protein, such as gelatin.

In another embodiment, the enzyme and substrate that produce a catalyst comprise lipase and lipid (for example, a triglyceride), protease and protein, trypsin and protein, chymotrypsin and protein, esterase and ester, lipase and ester, or esterase and triglyceride. In one embodiment, the enzyme is a lipase or esterase, optionally candida rugossa lipase, porcine liver esterase, Rhisopus oryzae esterase or Porcine pancrease lipase. In another embodiment, the substrate is a triglyceride or ester, optionally triacetin, tripropyrin, tributyrin, ethyl acetate, octyl acetate, butyl acetate or isobutyl acetate. In another embodiment, the enzyme and substrate that produce a catalyst comprise lactose dehydrogenase and lactose, papain and protein, pepsin and protein or pancreatin and soy protein.

The term “nitric oxide gas precursor” as used herein means any substrate that may be converted into nitric oxide gas. Accordingly, in an embodiment, the nitric oxide gas precursor is a substrate for enzymatic production of nitric oxide. In one embodiment, the nitric oxide gas precursor is L-arginine. In another embodiment, the nitric oxide gas precursor is nitrate or a salt thereof, such as potassium nitrate, sodium nitrate or ammonium nitrate or other nitrate. In one embodiment, the nitrate is nitrate produced from sweat. In yet another embodiment, the nitric oxide gas precursor is a nitrite or salt thereof, such as potassium nitrite or sodium nitrite. In one embodiment, 1-50 mmol of sodium nitrite are used. In another embodiment, 30 mmol of sodium nitrite are used. In yet another embodiment, the nitric oxide gas precursor is a nitric oxide donor, optionally nitroglycerin or isosorbide nitrate. In one embodiment, the enzyme comprises NiR and the nitric oxide gas precursor comprises potassium nitrite or the enzyme comprises NOS and the nitric oxide precursor comprises L-arginine. In another embodiment, the enzyme comprises a nitrate reductase and the nitric oxide gas precursor is a nitrate salt. In yet another embodiment, the nitric oxide gas precursor is a nitro-glycerine or nitrate located in an eluting transdermal system, such as a patch. In a further embodiment, the enzyme is glutathione S-transferase (GST) or cytochrome P450 system (P450) and the nitric oxide gas precursor is a nitroglycerine, a nitrosorbide dinitrate, or a nitrate.

Enzyme or catalyst activity is readily determined by an assay measuring the nitric oxide gas product. The preferred NO assay is a chemiluminescent assay. A sample containing nitric oxide is mixed with a large quantity of ozone. The nitric oxide reacts with the ozone to produce oxygen and nitrogen dioxide. This reaction also produces light (chemiluminescence), which can be measured with a photodetector. The amount of light produced is proportional to the amount of nitric oxide in the sample.

The disclosure also includes modified NOS and NIR polypeptides which have sequence identity of at least about: >20%, >25%, >28%, >30%, >35%, >40%, >50%, >60%, >70%, >80% or >90% more preferably at least about >95%, >99% or >99.5%, to SEQ ID NO:1 and SEQ ID NOs:2-5 (Table 1) respectively. Modified polypeptide molecules are discussed below.

Identity is calculated according to methods known in the art. Sequence identity is most preferably assessed by the BLAST version 2.1 program advanced search (parameters as above). BLAST is a series of programs that are available online from the National Center for Biotechnology Information (NCBI) of the U.S. National Institutes of Health. The advanced BLAST search is set to default parameters. (i.e. Matrix BLOSUM62; Gap existence cost 11; Per residue gap cost 1; Lambda ratio 0.85 default).

References to BLAST searches are: Altschul, S. F., Gish, W., Miller, W., Myers, E. W. & Lipman, D. J. (1990) “Basic local alignment search tool.” J. Mol. Biol. 215:403-410; Gish, W. & States, D. J. (1993) “Identification of protein coding regions by database similarity search.” Nature Genet. 3:266-272; Madden, T. L., Tatusov, R. L. & Zhang, J. (1996) “Applications of network BLAST server” Meth. Enzymol. 266:131-141; Altschul, S. F., Madden, T. L., Schäffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402; Zhang, J. & Madden, T. L. (1997) “PowerBLAST: A new network BLAST application for interactive or automated sequence analysis and annotation.” Genome Res. 7:649-656.

Preferably about: 1, 2, 3, 4, 5, 6 to 10, 10 to 25, 26 to 50 or 51 to 100, or 101 to 250 nucleotides or amino acids are modified. The disclosure includes polypeptides with mutations that cause an amino acid change in a portion of the polypeptide not involved in providing activity or an amino acid change in a portion of the polypeptide involved in providing activity so that the mutation increases or decreases the activity of the polypeptide.

In one embodiment, the enzyme has animal, plant, fungal or bacterial origin.

In another embodiment, the composition further comprises an enzyme cofactor. Enzyme cofactors useful in the device include tetrahydrobiopterin (H4B), calcium ions (Ca²⁺), flavin adenine dinucleotide (FAD), flavin mononuleotide (FMN), beta-nicotinamide adenine dinucleotide phosphate reduced (NADPH), molecular oxygen O₂ and calmodulin.

The compositions and devices described herein can be made more effective by the addition of bioactive molecules that react with reactive oxygen species (ROS) which normally consume nitric oxide. Bioactive low molecular weight (LMWT) and enzymatic antioxidants can prevent the consumption of NO by ROS (Serarslan et al. 2007). The reaction between NO and ROS forms peroxynitrite (ONO₂⁻), disabling NO and preventing its normal physiologic action. The use of antioxidants, either added pure or produced in an in-situ reaction between cell or enzyme isolates and substrate, can prevent the consumption of NO by ROS providing an improved NO delivery formulation for topical application in cosmesis.

Accordingly, in another embodiment, the composition further comprises an antioxidant for maintaining a reducing environment. The antioxidant may be expressed by the live cell or produced in a reaction between a second enzyme, either added or expressed by the live cell, and an antioxidant precursor. In one embodiment, the antioxidant is caffeic acid, ferulic acid, or chlorogenic acid. In another embodiment, the antioxidant is dithionite, methaquinone or ubiquinone. In yet another embodiment, the antioxidant is a vitamin, optionally, vitamin K, vitamin E or vitamin C.

The term “live cell” as used herein means any type of cell that is capable of converting nitric oxide precursor to nitric oxide at the site of action. In one embodiment, the cell is a human, bacterial or yeast cell. In another embodiment the cell is a probiotic microorganism of the genus Lactobacillus, Bifidobacteria, Pediococcus, Streptococcus, Enterococcus, or Leuconostoc. In one embodiment, the cell is Lactobacillus plantarum, Lactobacillus fermentum, Pediococccus acidilactici, or Leuconostoc mesenteroides. In another embodiment, the cell is a yeast cell selected from the group consisting of one or more of a Torula species, baker's yeast, brewer's yeast, a Saccharomyces species, optionally S. cerevisiae, a Schizosaccharomyces species, a Pichia species optionally Pichia pastoris, a Candida species, a Hansenula species, optionally Hansenula polymorpha, and a Klyuveromyces species, optionally Klyuveromyces lactis. In one embodiment, the cell is a bacteria that produces a mild acid, including, without limitation, lactic acid, acetic acid, malic acid and tartaric acid. In yet another embodiment, the cell is a lactic acid bacteria (LAB) or an acetobacter, such as acetobacter pastureianis.

In a further embodiment, the cell is a genetically engineered cell expressing an enzyme that is capable of converting a nitric oxide precursor to nitric oxide gas. In one embodiment, the cell is a genetically engineered yeast expressing NOS or NiR enzyme. In another embodiment, the cell is a genetically engineered bacteria expressing NOS or NiR enzyme. In yet another embodiment, the cell is Escherichia coli BL21 (nNOSpCW), an E. coil or Lactobacillus strain expressing bacterial nitrite reductases, optionally a copper-dependant nitrite reductase from Alcaligenes faecalis S-6 or an E. coli or Lactobacillus strain expressing a cytochrome cd1 nitrite reductase from Pseudomonas aeruginosa.

A person skilled in the art would be able to quantify the amount of NO produced by a cell or enzyme. For example, Kikuchi et al. describe a method for the quantification of NO using horseradish peroxidase in solution (Kikuchi et al., 1996). Archer et al. reviewed the measurement of NO in biological systems and found that the chemiluminescence assay is the most sensitive technique with a detection threshold of roughly 20 pmol (Archer 1993; Michelakis and Archer, 1998).

In another embodiment, the cell is microencapsulated. In one embodiment, the microcapsule comprises Alginate/Poly-I-lysine/Alginate (APA), Alginate/Chitosan/Alginate (ACA), or Alginate/Genipin/Alginate (AGA) membranes. In another embodiment, the microcapsule comprises Alginate/Poly-I-lysine/Pectin/Poly-I-lysine/Alginate (APPPA), Alginate/Poly-I-lysine/Pectin/Poly-I-lysine/Pectin (APPPP), Alginate/Poly-L-lysine/Chitosan/Poly-I-lysine/Alginate (APCPA), alginate-polymethylene-co-guanidine-alginate (A-PMCG-A), hydroxymethylacrylate-methyl methacrylate (HEMA-MMA), Multilayered HEMA-MMA-MAA, polyacrylonitrilevinylchloride (PAN-PVC), acrylonitirle/sodium methallylsuflonate (AN-69), polyethylene glycol/poly pentamethylcyclopentasiloxane/polydimethylsiloxane (PEG/PD5/PDMS) or poly N,N-dimethyl acrylamide (PDMAAm) membranes. In a further embodiment, the microcapsule comprises alginate, hollow fiber, cellulose nitrate, polyamide, lipid-complexed polymer, a lipid vesicle a siliceous encapsulate, cellulose sulphate/sodium alginate/polymethylene-co-guanidine (CS/A/PMCG), cellulose acetate phthalate, calcium alginate, k-carrageenan-Locust bean gum gel beads, gellan-xanthan beads, poly(lactide-co-glycolides), carageenan, starch polyanhydrides, starch polymethacrylates, polyamino acids or enteric coating polymers.

In another embodiment, the cell or enzyme of the composition is immobilized in a reservoir, such as a slab. In one embodiment, the reservoir or slab comprises a polymer. In a particular embodiment, the polymer is a natural polymer such as alginate, chitosan, agarose, agaropectin, or cellulose.

In yet another embodiment, the composition further comprises growth media for cells. Typical growth media include MRS broth, LB broth, glucose, or carbon source containing growth media. The choice of growth media depends on the requirements of the particular cells of the composition of the device of the application.

In a further embodiment, a reducing agent is added. In one embodiment, the reducing agent leads to improved stoichiometry and additional NO production. In an embodiment, the reducing agent is sodium iodide (NaI).

In a further embodiment, the device or composition comprises a nitric oxide gas or radical concentrating agent. The term “nitric oxide gas or radical concentrating agent” as used herein is intended to cover any substance that is capable of collecting and concentrating the nitric oxide gas for application to the skin.

In one embodiment, the nitric oxide gas or radical concentrating agent comprises lipid or lipid-like molecules. The term “lipids and lipid-like molecules” as used herein mean substances that are fat soluble. An example of a lipid-like molecule is a lipopolysaccharide which is a lipid and a carbohydrate molecule joined by a covalent bond.

In another embodiment, the nitric oxide gas or radical concentrating agent comprises hydrocarbon or hydrocarbon-like molecules. The term “hydrocarbon” as used herein means a hydrogen and carbon containing compound which has a carbon “backbone” and bonded hydrogens, sulfur or nitrogen (impurities), or functional groups. The term “hydrocarbon-like molecule” refers to a molecule that has a carbon backbone and contains hydrogens but may have a complex and highly bonded or substituted structure. Both hydrocarbons and hydrocarbon-like molecules are lipid soluble.

In yet another embodiment, the nitric oxide gas or radical concentrating agent comprises a spacer, a gas cell containing structure or a sponge.

In one aspect, the nitric oxide gas precursor and the composition comprising live cell, enzyme or catalyst are separated until use. Accordingly in one embodiment of the composition of the application, the nitric oxide gas precursor and composition comprising live cell, enzyme or catalyst are kept separate and are mixed immediately prior to use. In an embodiment of the device, the active layer and reservoir layer are separated by a separator. The separator is a physical barrier, optionally made from plastic or other suitable material, typically between the active layer and reservoir layer, that prevents the contents of the active layer and reservoir layer from combining. In another embodiment, the casing further comprises at least one valve connecting the active layer and the reservoir layer, wherein the valve has an initial closed position in which the cell or enzyme are separate from the precursor and an open position in which the active layer and reservoir layer are in fluid communication, and the cell or enzyme precursor are permitted to flow between the layers. In another embodiment, the valve comprises a one-way valve, and wherein in the open position either the enzyme or cell or the precursor is permitted to flow between the layers. In another embodiment, the valve comprises a pressure actuated valve that is actuable from the closed position to the open position by compression of the device, optionally manual compression. In yet a further embodiment, the composition alone or in the device is dehydrated and is inactive until hydration.

### Methods and Uses

In another aspect, the application provides the use of a device or composition of the application for skin cosmesis in a subject in need thereof. In another embodiment, the application provides methods for skin cosmesis in a subject in need thereof using a device or composition of the application. In a further embodiment, the application provides the use of a composition or device of the application for skin cosmesis in a subject in need thereof.

The term “skin cosmesis” as used herein means the preservation, restoration or bestowing of beauty (i.e. improved appearance) to skin and includes, without limitation, at least one of the following results: increased extracellular matrix deposition, increased moisture content, improved blood flow, improved elasticity, increased antioxidant capacity, improved orderly collagen deposition, and reduced inflammation of skin surface. Various techniques are known in the art for determining preservation, restoration and improved appearance of skin, such as morphologic evaluations of skin at periodic intervals to assess texture, dryness, skin tone, color and/or other skin attributes. In one embodiment, the methods described herein are for improving blood flow to the skin. In another embodiment, the methods described herein are for improving orderly collagen deposition. “Skin cosmesis” also includes, without limitation, rejuvenating the skin, firming the skin, improving the extracellular matrix, reducing wrinkles, resurfacing the skin, improving skin hydration, increasing thickness of dermis, improving disscolouration of skin, improving integument cosmesis, such as nail cosmesis or inhibiting or increasing hair growth.

The term “subject” as used herein means an animal, optionally a mammal and typically a human.

In one aspect, the device or composition is kept inactive until the time of application onto the skin, for example, by keeping the nitric oxide gas precursor and composition comprising the live cell, enzyme or catalyst separate, such as two creams, emulsions and/or gels, or by dehydrating the composition until use, such as with a powder composition or dissolvable film. Accordingly, in one embodiment, the application provides a method for skin cosmesis in a subject in need thereof comprising:

contacting the skin with a casing permeable to nitric oxide gas, the casing containing a plurality of inactive agents that, when activated, react to produce nitric oxide gas; and

activating the inactive agents to produce nitric oxide gas,

wherein the nitric oxide gas communicates through (i.e. passes through) the casing and contacts the skin for cosmesis in the subject in need thereof.

In another embodiment, the application provides a method for skin cosmesis in a subject in need thereof comprising providing inactive agents that, when activated, react to produce nitric oxide gas; activating the inactive agents to produce nitric oxide gas; and applying the activated agents to the skin of the subject.

In one embodiment, the inactive agents comprise i) a nitric oxide gas precursor, and ii) (a) an isolated enzyme or a live cell expressing an endogenous enzyme, the enzyme (i) having activity that converts the nitric oxide gas precursor to nitric oxide gas or (ii) having activity on a substrate that produces a catalyst that causes the conversion of the nitric oxide gas precursor to nitric oxide gas or (b) a live cell expressing a catalyst for converting the nitric oxide gas precursor to nitric oxide gas.

In another embodiment, the inactive agents comprise separated agents and activating the inactive agents comprise combining the separated agents. In one embodiment, the separated agents are combined by applying pressure or temperature to the device or composition. In another embodiment, the inactive agents comprise dehydrated agents and activating the inactive agents comprise hydration.

In yet another embodiment, there is provided a method for skin cosmesis in a subject in need thereof comprising:

contacting the skin with a nitric oxide gas releasing composition or device, the composition or device comprising an isolated enzyme or a live cell expressing an endogenous enzyme, the enzyme (i) having activity that converts nitrate to nitric oxide gas or (ii) having activity on a substrate that produces a catalyst that causes the conversion of nitrate to nitric oxide gas or (b) a live cell expressing a catalyst for converting nitrate to nitric oxide gas;

wherein the composition reacts with nitrate in sweat on the skin to produce nitric oxide gas for cosmesis in the subject in need thereof.

In a further embodiment, the device or composition is applied to the skin for a treatment period without inducing toxicity to the subject or skin. The treatment period will depend on the type of device or composition used. For example, for a device described herein, the treatment period typically is from about 1 to 24 hours, preferably about 6-10 hours and more preferably about 8 hours. For a composition contained in a patch, the treatment period typically is from about 1 to 8 hours. For a cream composition, the cream is typically applied one to three times daily. For a mask composition, the treatment period is typically from about 1 to 8 hours, optionally, 1-2 hours.

In yet a further embodiment, the NO is produced by the device or composition from about 3 to 160 parts per million volume (ppmv), optionally from about 25 to 125 ppmv.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Nitric Oxide Gas Production

### Results:

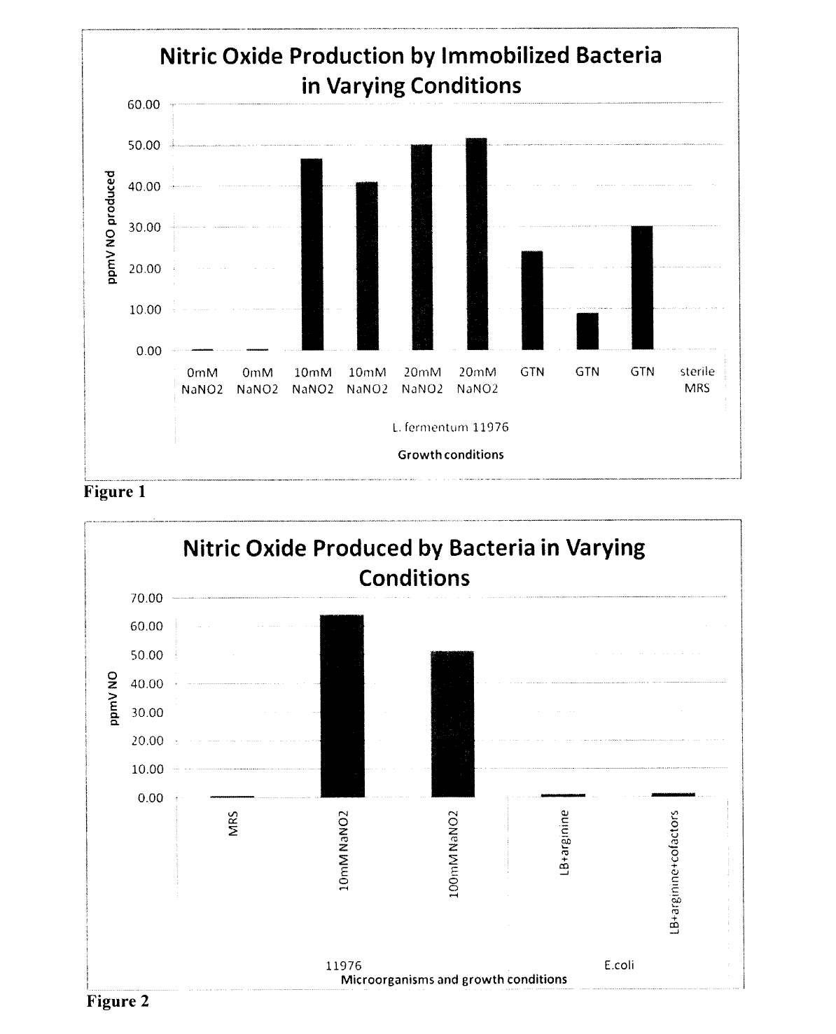

Tables 2-4 show the reaction that produces nitric oxide from a precursor. The results show that live bacteria are able to produce nitric oxide gas (gNO) when immobilized in a slab-like piece of agarose supplemented with MRS growth media and either nitrite or a nitroglycerine patch (FIG. 1). The results in FIG. 2 show that live bacteria are able to produce nitric oxide gas when grown in media with the indicated cofactors. Without wishing to be bound by theory, the most probable mechanism for nitric oxide production from nitrite is the reduction of the salt to gNO by lactic acid produced by the metabolically active bacteria. The most probable mechanism of gNO production from nitroglycerine is that the organisms produce lactic acid which reduces nitroglycerine to nitrite and the resulting nitrite is reduced to nitric oxide again by lactic acid. In this way, the immobilized bacteria are capable of releasing gNO from a device or composition of the disclosure and onto skin over a period of time and in proportion to their metabolic activity.

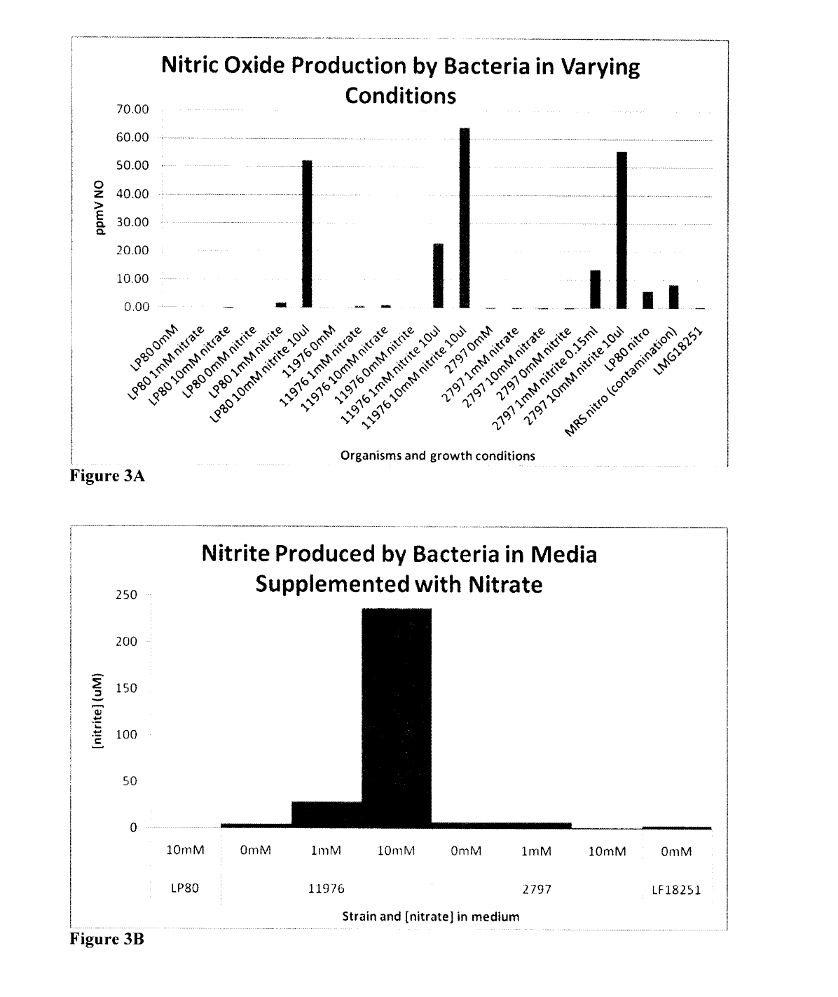

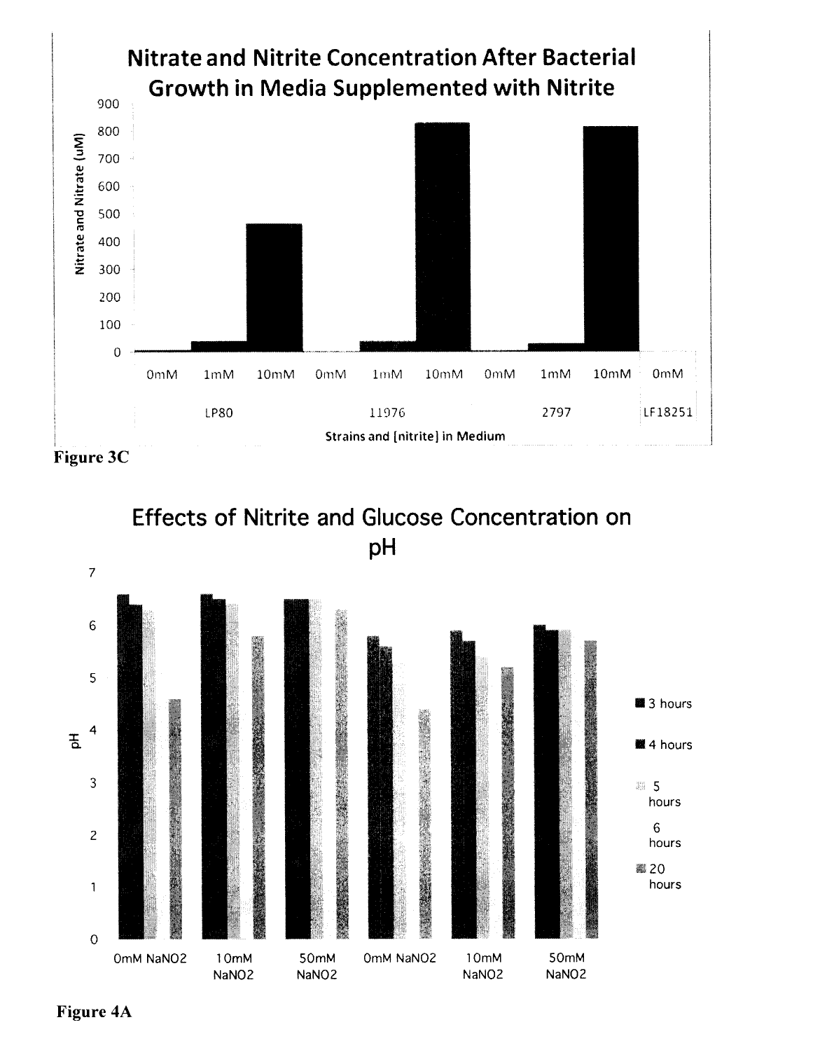

Nitrite salts can be reduced to gNO by several different lactic acid producing bacteria (LAB) and the quantity of gNO produced depends on the concentration of nitrite substrate and the acid producing capability of the bacteria (FIG. 3A). Some bacteria such as Lactobacillus fermentum (ATCC 11976) have a nitrate reducing capacity and hence nitrates, such as potassium nitrate, can be used as substrate for the production of gNO by these bacteria. The nitrate substrate can be converted to nitrite which can then be reduced to gNO by lactic acid produced by the bacteria (FIG. 3B). Again, this example substantiates the use of nitrates, nitrites, or some other nitric oxide precursor as a substrate with live cells or enzymes in a device or composition for skin cosmesis.

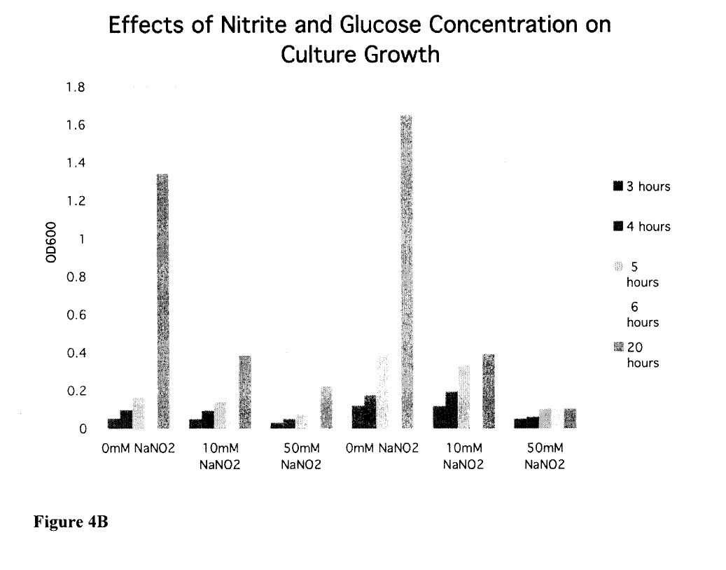

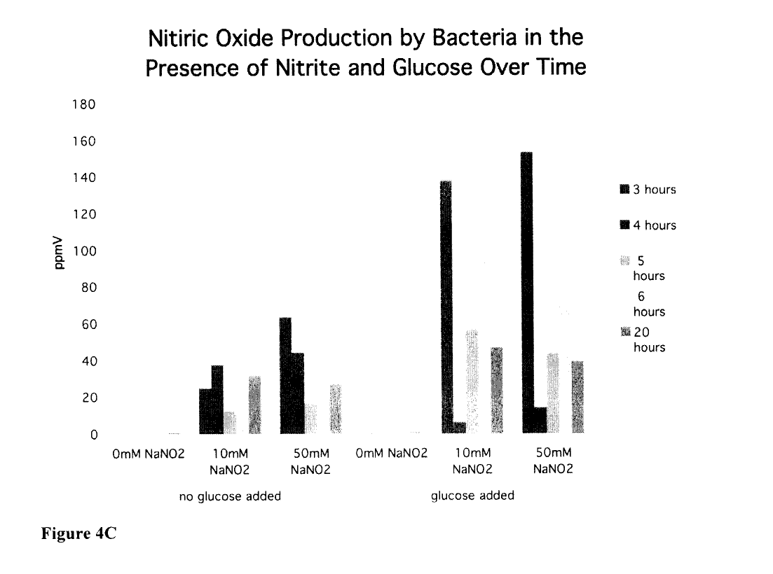

The addition of glucose to growth media containing LAB results in increased acidification of the growth media over time (lower pH). When supplemented with glucose, lower pH values were achieved with Lactobacillus fermentum (ATCC 11976) over time (FIG. 4A). The addition of nitrite to the growth media, although making more substrate available for the production of gNO, inhibited the growth of bacteria as seen by reduced OD600 values (FIG. 4B). Increased concentrations of lactic acid (lower pH values) were observed in media supplemented with glucose and despite the inhibition of bacterial growth at higher concentrations of nitrite, an increased capacity for reduction and more gNO was produced by bacteria in growth media supplemented with both glucose and nitrite (FIG. 4C). A pattern of increasing and decreasing gNO concentrations was seen. The interplay between LAB, growth media, glucose, NO substrate, NO, and lactic acid provides a useful therapeutic system for topical application. The continued release of gNO by immobilized or microencapsulated live cells or enzymes over the entire therapeutic duration is very advantageous for this cell/enzyme based technology.

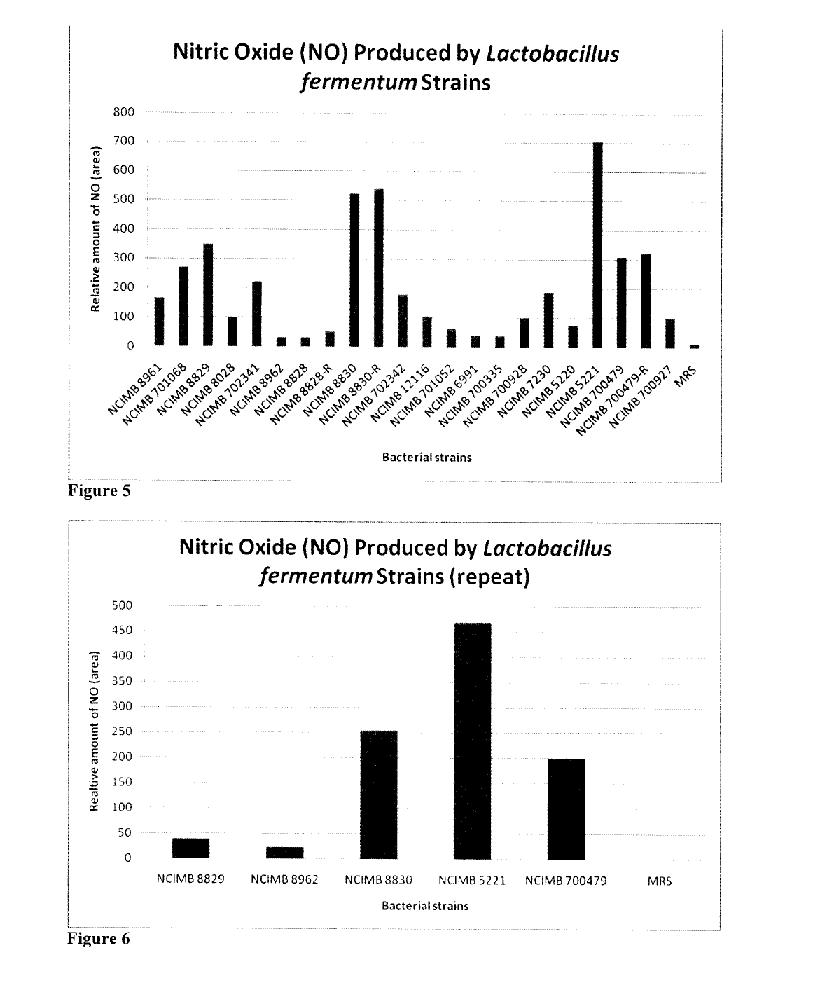

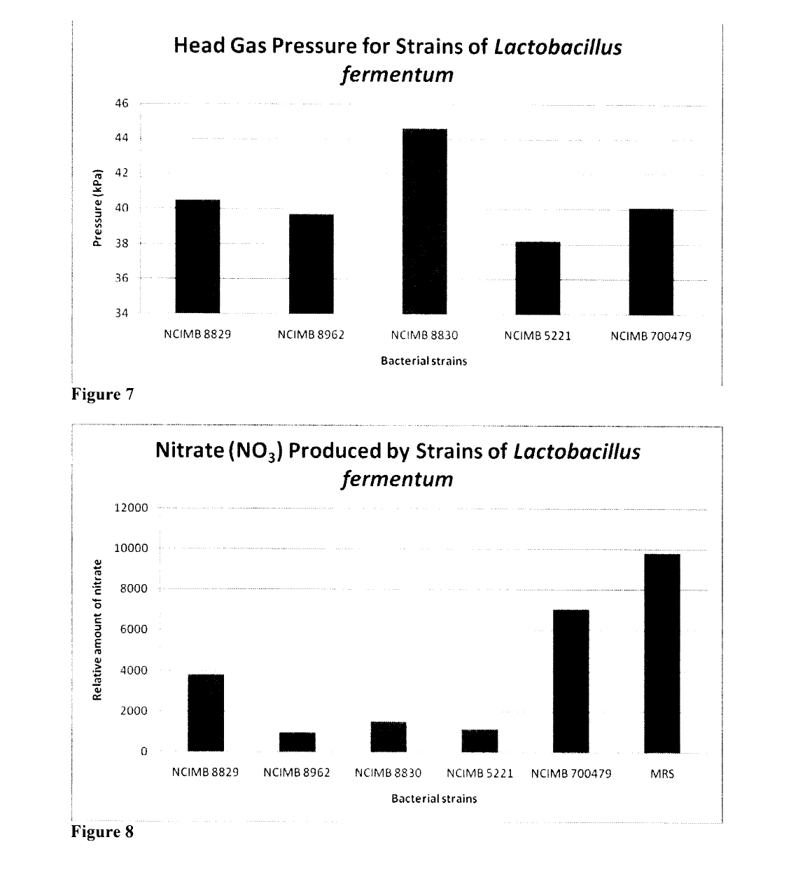

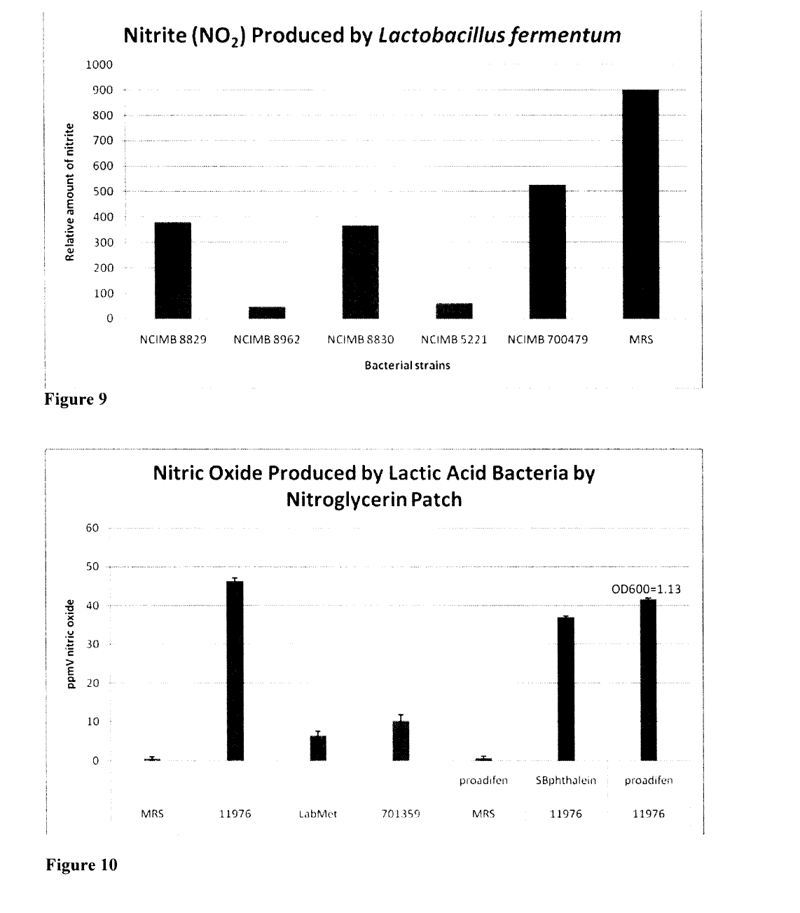

The results also show that some strains of Lactobacillus are capable of producing nitric oxide when grown in MRS broth (FIG. 5 and FIG. 6). The head gas pressure was also measured in the vessel where the bacterial strains were grown (FIG. 7). The present inventors have also shown the ability of the bacterial strains to produce nitrate and nitrite after growth in media for 20 hours (FIGS. 8 and 9). Nitric oxide is also produced from lactic acid bacteria by a use of a nitroglycerin patch (FIG. 10).

Live Cell or Enzyme Having Activity that Produces a Catalyst

A crude extract of pancreatic enzyme (5% pancreatin) is optionally immobilized in a slow gelling hydropolymer of alginate (2% alginic acid, sodium pyrophosphate, calcium sulphate, water) with a protein/lipid containing substrate (1% soy protein isolate) and a nitric oxide donor salt (NaNO2). Alternatively, a reducing agent such as sodium iodide (NaI) is optionally used to improve the stoichiometry of the reaction and provide the added bactericidal effects of iodine gas. This device or patch is typically lyophilized and stored for later use. Once made active by the addition of water and with a gas impermeable and optionally adhesive backing and a gas permeable but protective skin interface (or contact surface), is useful to produce high or low therapeutic levels of nitric oxide gas for topical application to skin.

### Materials and Methods:

### NO Gas Production by Immobilized Bacteria in Varying Conditions (FIG. 1)

MRS agar (Fisher scientific) was autoclaved in a Wheaton bottle (Fisher scientific) capped with a septum-equipped PTFE cap. Once the agar was cooled, but still liquid, sodium nitrite (Sigma-Aldrich) was added to the desired final concentration from a sterile 1M stock. Alternatively, a Nitro-Dur 0.8 transdermal nitro-glycerine patch (Key pharmaceuticals) was introduced in the bottle. An overnight culture of Lactobacillus fermentum (ATCC 11976) (OD600=2) was used to aseptically inoculate the agar to a 1:50 dilution. The agar was left to harden at room temperature for 30 minutes and then incubated for 20 hours at 37° C. A 100 μL syringe (Hamilton) was used to remove gas from the headspace and to inject it in the injection port of a chemiluminescence NO analyzer (Sievers®, GE analytical). The area under the curve for each injection was recorded and the parts per million by volume value was calculated using a pre-determined conversion factor.

Growth of Lactobacillus fermentum (ATCC 11976) (FIG. 2)

MRS broth (Fisher scientific) was autoclaved in a Wheaton bottle (Fisher scientific) capped with a septum-equipped PTFE cap. Sodium nitrite (Sigma-Aldrich) was added to the desired final concentration from a sterile 1M stock. An overnight culture of Lactobacillus fermentum (ATCC 11976) (OD600=2) was used to aseptically inoculate the broth to a 1:50 dilution. After 20 hours at 37° C., a 100 μL syringe (Hamilton) was used to remove gas from the headspace and to inject it in the injection port of a chemiluminescence NO analyzer (Sievers®, GE analytical). The area under the curve for each injection was recorded and the parts per million by volume value was calculated using a pre-determined conversion factor.

Growth of Escherichia coli BL21 (pnNOS) (pGroESL) (FIG. 2)

An E. coli strain harboring a plasmid encoding the rat neuronal nitric oxide synthase (pnNOS) and a plasmid encoding chaperone proteins (pGroESL) was grown for 20 hours in LB containing 100 μg/ml ampicillin and 10 μg/ml chloramphenicol. 1 mM arginine was added and the cofactors required for neuronal nitric oxide synthase activity (12 μM BH4, 120 μM DTT and 0.1 mM NADPH) were added to one of the cultures. Sampling of the head gas was done as described above.

### Nitric Oxide Production by Bacteria in Varying Conditions (FIG. 3)

MRS broth (Fisher scientific) was autoclaved in a Wheaton bottle (Fisher scientific) capped with a septum-equipped PTFE cap. Sodium nitrite (Sigma-Aldrich) was added to the desired final concentration from a sterile 1M stock. An overnight culture of Lactobacillus fermentum (ATCC 11976), Lactobacillus plantarum LP80, Lactobacillus fermentum NCIMB 2797 or Lactobacillus fermentum (LMG 18251) (OD600=2) was used to aseptically inoculate the broth to a 1:50 dilution. After 20 hours at 37° C., a 100 μL syringe (Hamilton) was used to remove gas from the headspace and to inject it in the injection port of a chemiluminescence NO analyzer (Sievers®, GE analytical). The area under the curve for each injection was recorded and the parts per million by volume value was calculated using a pre-determined conversion factor.

### Nitrite Measurements (FIG. 3)

Nitrite levels were measured by injecting 1 ml of the growth medium in the reaction vessel of the chemiluminescence NO analyzer (Sievers®, GE analytical) containing 3 ml glacial acetic acid and 1 ml 50 mM KI. Reaction of the nitrite with the acid and the KI releases NO gas which is in turn detected by the analyzer.

### Nitrate Measurements (FIG. 3)

Nitrate levels were measured by injecting 1 ml of the growth medium into the reaction vessel of the chemiluminescence NO analyzer (Sievers®, GE analytical) containing 3 ml 1M HCl and 50 mM VCl₃. The reaction was performed at 95° C. using the heating water bath and pump to heat the reaction vessel to 95° C. Reaction of the nitrate in the sample with the acid and the VCl₃ releases NO gas which is in turn detected by the analyzer.

### Nitric Oxide Production by Bacteria in the Presence of Nitrite and Glucose Over Time (FIG. 4)

MRS broth (Fisher scientific) with the required amount of glucose (20 g/L or 100 g/L) was autoclaved in a Wheaton bottle (Fisher scientific) capped with a septum-equipped PTFE cap. Sodium nitrite (Sigma-Aldrich) was added to the desired final concentration from a sterile 1M stock. An overnight culture of Lactobacillus fermentum 11976 (OD600=2) was used to aseptically inoculate the broth to a 1:50 dilution. After growth at 37° C. for the required amount of time without shaking, a 1 ml syringe equipped with a 27G 1.25″ needle was used to puncture the septum and remove 0.7 ml of the medium. This aliquot was used to perform pH (FIG. 4A) and spectrophotometric (FIG. 4B) measurements. The septum was then punctured with a 100 μL syringe (Hamilton) to remove gas from the headspace and injected in the injection port of a chemiluminescence NO analyzer (Sievers®, GE analytical). The area under the curve for each injection was recorded and the parts per million by volume value was calculated using a pre-determined conversion factor (FIG. 4C).

Nitric Oxide Production by Lactobacillus fermentum (FIGS. 5-9)

Strains of Lactobacillus fermentum (NCIMB, Scotland) were grown for 20 hours in a septum-equipped bottle containing 20 ml of MRS broth. The pressure in the bottle resulting from gas production was measured using a manometer (Fisher scientific) equipped with a needle to puncture the septum. 1 ml of head gas was withdrawn and injected in a nitric oxide analyzer (Seivers, General Electric) and the area under the curve was reported as a representation of the relative amount of nitric oxide gas present in the headspace. 10 ul of the medium was subsequently withdrawn and injected in the analyzer with glacial acetic acid and excess sodium iodide present in the injection chamber. This resulted in the nitrite being converted to nitric oxide gas which is then measured by the analyzer and reported as the relative amount of nitrite in the growth medium. The same process was repeated for the measurement of nitrate in the growth medium except that 1M HCl and excess vanadium chloride was present in the injection chamber to convert the nitrate in the medium to nitric oxide gas. The gas thereby measured by the analyzer gave a relative measure of the amount of nitrate in the growth medium.

### Nitric Oxide Produced by Lactic Acid Bacteria by Nitroglycerin Patch (FIG. 10)

MRS agar (Fisher scientific) was autoclaved in a Wheaton bottle (Fisher scientific) capped with a septum-equipped PTFE cap. Once the agar was cooled, but still liquid, a Nitro-Dur 0.8 transdermal nitroglycerin patch (Key pharmaceuticals) was introduced in the bottle. An overnight culture of Lactobacillus reuteri (NCIMB 701359), Lactobacillus reuteri (LabMet) or Lactobacillus fermentum (ATCC 11976) (OD600=2) was used to aseptically inoculate the agar to a 1:50 dilution. Proadifen (SKS-525A), an inhibitor of the P450 enzyme was added to a final concentration of 50 μM from a 64 mM stock in water and sulfobromophthalein, an inhibitor of gluthathione-S-transferase, was added to a concentration of 1 mM from a 30 mM stock in water. The agar was left to harden at room temperature for 30 minutes and then incubated for 20 hours at 37° C. A 100 μL syringe (Hamilton) was used to remove gas from the headspace and to inject it in the injection port of a Sievers NO analyzer (GE analytical). The area under the curve for each injection was integrated and recorded and the parts per million by volume value was calculated using a pre-determined conversion factor.

### Devices

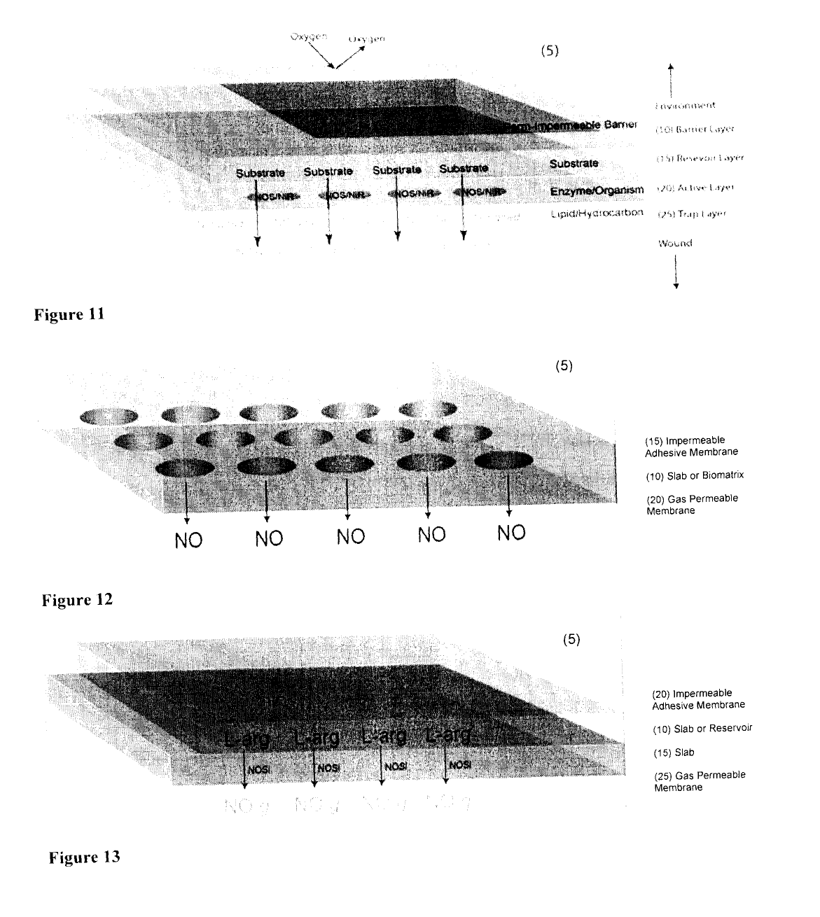

FIGS. 11-14 provide examples of devices that are used to provide a source of nitric oxide to the skin.

FIG. 11 shows a multilayered nitric oxide producing device (5) made up of a barrier (10), reservoir (15), active (20), and trap layer (25) as one proceeds from the environment to the skin. The barrier layer (10) maintains variable permeability to oxygen while protecting the skin and adhering the patch. The reservoir layer (15) contains substrate, such as potassium nitrite or arginine, for the enzyme in the active layer. The active layer (20) contains enzyme producing microorganisms or free enzyme and cofactors for the production of nitric oxide. The trap layer (25) is made up of lipids or hydrocarbons for concentrating nitric oxide radicals nearest the skin.

FIG. 12 shows a single layered device (5) with NO producing bacteria immobilized in polymer slab or biomatrix (10) for the production of NO for topical application to skin. The production of NO is maintained by the immobilized cells and protected from contact with O₂ by an impermeable adhesive membrane (15) above the immobilized bacteria. Also, the transmission of other biologic material can be prevented from coming into contact with the skin by a gas permeable membrane (20).

FIG. 13 shows a simple layered medical device (5) with L-arginine immobilized in slab or in a reservoir (10) above NOS enzyme immobilized in a slab (15) for the production of NO for the topical application to skin. The production of NO is maintained by the immobilized cells and protected from contact with O₂ by an impermeable adhesive membrane (20) above the immobilized bacteria. Also, the transmission of other biologic material can be prevented from coming into contact with the skin by a gas permeable membrane (25).

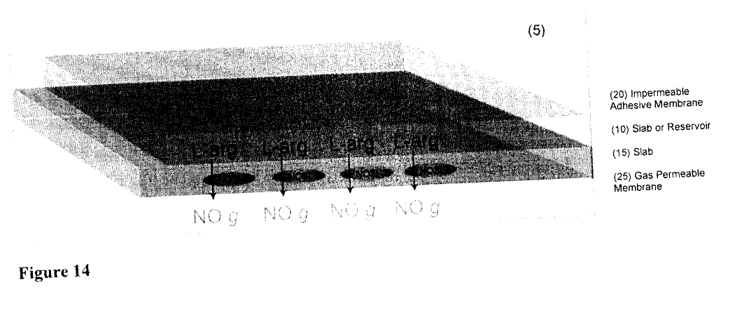

FIG. 14 shows a simple layered medical device (5) with L-arginine immobilized in slab or in a reservoir (10) above NOS producing bacteria immobilized in an alginate slab (15) for the production of NO for topical application to skin. The production of NO is maintained by the immobilized cells and protected from contact with O₂ by an impermeable adhesive membrane (20) above the immobilized bacteria. Also, the transmission of other biologic material can be prevented from coming into contact with the skin by a gas permeable membrane (25).

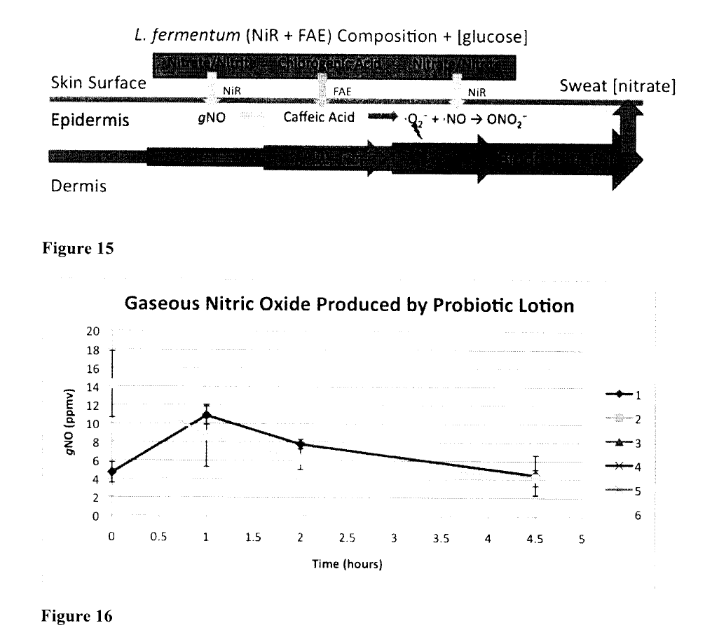

FIG. 15 shows a schematic of a composition comprising probiotic bacteria expressing NiR and FAE active L. fermentum and glucose substrate. The ferulic acid esterase (FAE) and nitrate reductase (NiR) active bacteria produce caffeic acid (CA) antioxidant from chlorogenic acid substrate and nitric oxide gas (gNO) from nitrate (NO₂⁻) or nitrite (NO₃⁻) in the composition or from sweat nitrate. The bioactive antioxidant produced can act to neutralize reactive oxygen species (ROS) present in epidermis and dermis, reducing oxidative damage and causing the cosmesis of treated skin. The caffeic acid antioxidant can also prevent consumption of gNO by ROS by a process that produces peroxynitrite (ONO₂⁻), increasing the activity of NO on treated skin.

### Compositions

### Lotion Formulation:

### Materials and Methods:

A vehicle composed of 0.1 mL Polysorbate 80, 2 mL of glycerol, 6.4 mL sterile distilled water and 1.5 mL rice bran oil was mixed by addition of components under constant stirring followed by sonication for 5 minutes, under constant duty cycle at the maximum microtip output using a Bronson sonifier to form an emulsion.

Active forms were made by replacing distilled water with 30 mM sodium nitrite and/or 10% glucose solutions. Immediately prior to use 100 mg lyophilized L. fermentum NCIMB 7230 microcapsules were added to the 1 mL portions of the final mixtures.

Nitric oxide measurements were made by enclosing a 1 mL portion of lotion in a gas impermeable pouch with small perforations near the top that were further enclosed in a sealed gas impermeable chamber. Gas was sampled through a septum hourly and NO concentrations were measured with a chemiluminescence NO analyzer (Sievers).

### Results:

Results of gaseous nitric oxide produced by the probiotic solution are shown in FIG. 16. Vehicle as described was supplemented with: (1) 30 mM NaNO₂, 10% glucose and lyophilized, microencapsulated L. fermentum NCIMB 7230 (100 mg/mL); (2) 30 mM NaNO₂, 10% glucose; (3) lyophilized, microencapsulated L. fermentum NCIMB 7230; (4) vehicle alone; (5) 30 mM NaNO₂ and (6) 30 mM NaNO₂ and lyophilized, microencapsulated L. fermentum NCIMB 7230. These mixtures were sealed in air tight chambers and gas produced sampled hourly through a septum and measured with chemiluminescence NO analyzer (Sievers).

### Cream Formulation:

### Materials and Methods:

A two component face cream was made with component A consisting of a cream composed of: 3.5 g ascorbic acid-6-palmitate, 6.3 mL glycerol, 0.1 mL polysorbate 80 and 10 mg/ml lyophilized L. fermentum NCIMB 7230 microcapsules and component B which consisted of: 7 mL of a 30 mM sodium nitrite 1% w/v sodium alginate gel polymerized with 10 mM calcium chloride.

Before use component A and B were stirred together manually until an even consistency was achieved. 100 μL samples were placed in 2 mL vials with septum caps and head gas was sampled and nitric oxide (gNO) was measured with a chemiluminescence NO analyzer (Sievers) hourly for four hours and again after 70 hours.

The effect of individual components was determined by comparing the nitric oxide produced by vehicle alone (no ascorbic acid-6-palmitate, sodium nitrite or lyophilized L. fermentum NCIMB 7230), to vehicle with 30 mM sodium nitrite, vehicle with L. fermentum NCIMB 7230, vehicle with ascorbic acid-6-palmitate, vehicle with 30 mM sodium nitrite and L. fermentum NCIMB 7230, vehicle with ascorbic acid-6-palmitate and 30 mM sodium nitrite, vehicle with Lyophilized L. fermentum NCIMB 7230 microcapsules and ascorbic acid-6-palmitate and final formula cream.

To determine if the nitric oxide production seen with sodium nitrite and ascorbic acid-6-phosphate was due to the acidity of ascorbic acid-6-palmitate a lotion composed of vehicle with ascorbic acid-6-palmitate and 30 mM sodium nitrite and the pH was measured. Another sample was adjusted to pH 8 with sodium hydroxide and the NO production was measured as above.

### Results:

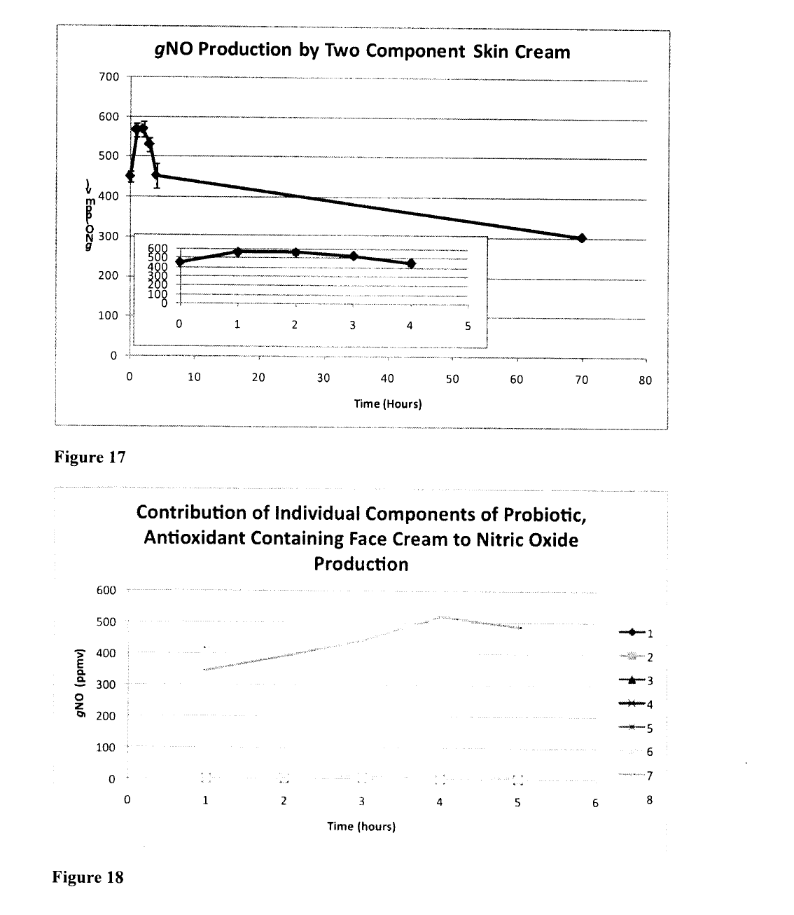

Results of gaseous nitric oxide (gNO) produced by the two component antioxidant skin cream containing probiotic bacteria are shown in FIG. 17. A non-aqueous phase containing ascorbic acid-6-palmitate, lyophilized L. fermentum NCIMB 7230 microcapsules was mixed with an aqueous gel containing 30 mM sodium nitrite and a 100 μL aliquot was place in a sealed 2 mL vial kept at room temperature and the head gas was sampled hourly for four hours and again after 70 hours and nitric oxide concentration was determined by measurements with a chemiluminescence NO analyzer (Sievers).

The results of contribution of individual components of the probiotic, antioxidant containing face cream to nitric oxide production is shown in FIG. 18. (1) 100 μL aliquots of vehicle alone (no ascorbic acid-6-palmitate, sodium nitrite or lyophilized L. fermentum NCIMB 7230 microcapsules), (2) vehicle with ascorbic acid-6-palmitate, (3) vehicle with lyophilized L. fermentum NCIMB 7230 microcapsules, (4) vehicle with lyophilized microcapsules and ascorbic acid-6-palmitate, (5) vehicle with 30 mM sodium nitrite, (6) vehicle with 30 mM sodium nitrite and L. fermentum NCIMB 7230, (7) vehicle with ascorbic acid-6-palmitate and 30 mM sodium nitrite, and (8) vehicle containing ascorbic acid-6-palmitate, sodium nitrite and lyophilized L. fermentum NCIMB 7230 microcapsules were placed in sealed 2 mL vials. The head gas was sampled hourly and nitric oxide content determined using a chemiluminescence NO analyzer (Sievers).

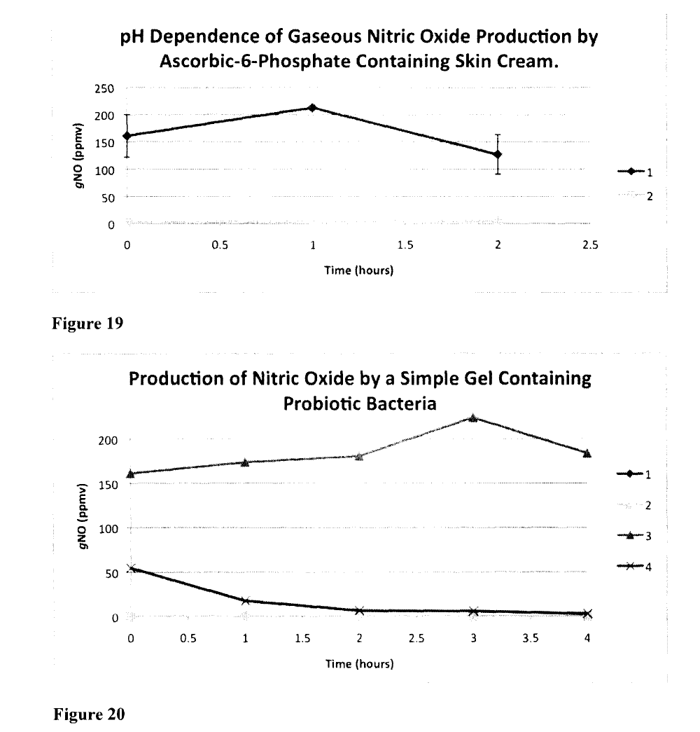

The results of pH dependence of gaseous nitric oxide (gNO) production by ascorbic-6-phosphate containing skin cream is shown in FIG. 19. The pH of nitric oxide producing cream containing ascorbic acid-6-palmitate was found to be six. (1) A 100 μL sample of pH 6 ascorbic acid-6-phosphare cream was sealed in a 2 mL vial in parallel with (2) a sample with a pH adjusted to 8 with sodium hydroxide. The head gas was sampled every 30 minutes and nitric oxide content determined using a chemiluminescence NO analyzer (Sievers).

### Gel Formulation:

### Materials and Methods:

A gel composed of an autoclaved solution of 30 mM NaNO2, 10% w/v glucose, 5% (w/v) alginate was prepared and 10% v/v L. fermentum NCIMB 7230 culture was added to an aliquot of this. A 100 μL aliquot of the gel with bacteria, a 100 μL aliquot of the gel without bacteria and 100 μL aliquot of the gel with distilled water added in place of 30 mM sodium nitrite with bacteria were sealed in 2 mL vials. In parallel, a 100 μL aliquot of the gel containing bacteria was also applied topically to a volunteer under a gas impermeable tape with a spacer to create a space for head gas. These experiments were run in parallel and head gas sample were taken hourly and nitric oxide concentration was measured with a chemiluminescence NO analyzer (Sievers).

### Results:

Results of the production of nitric oxide by the simple gel containing probiotic bacteria are shown in FIG. 20. A 100 μL aliquot of a 1% alginate, 30 mM sodium nitrite gel was supplemented with 10% v/v L. fermentum NCIMB 7230 culture was sealed in a 2 mL vials and compared to a gel without added bacteria and a gel without nitrite. In parallel, a 100 μL aliquot of the gel containing bacteria was also applied topically to a volunteer under a gas impermeable tape with a spacer to create a space for head gas. Head gas samples were taken hourly and nitric oxide concentration was measured with a chemiluminescence NO analyzer (Sievers).

### Mask Formulation:

### Materials and Methods:

A mask powder containing 5.5 g filler (diatomacious earth), 2.0 g Brace G alginate, 0.15 g sodium pyrophosphate, 2.2 calcium sulphate and 0.12 g sodium nitrite was made. 1 g of this powder was mixed with 1 g lyophilized L. fermentum NCIMB 7230 and hydrated with 6 L of distilled water. Two 100 μL aliquots of this mix was placed in 2 mL vials and compared to equivalent samples without added bacteria. Head gas was sampled every 30 minutes and the nitric oxide content was determined using a chemiluminescence NO analyzer (Sievers).

In vivo NO production was determined by placing 100 μL aliquots of mask with or without bacteria as above on the arms of two volunteers under gas impermeable tape with a spacer to create head gas space. Head gas was sampled at 30-minute intervals and measured every thirty minutes for two hours.

Blood flow changes were measured using a MoorVMS-LDF1 laser Doppler blood flow meter. An area was marked and a three-minute baseline blood flow value was recorded. The marked area and surrounding skin was then covered with a mask containing lyophilized L. fermentum NCIMB 7230 hydrated with 6 volumes of water for three minutes. Then the marked area was exposed and the probe replaced while the surrounding area was still under treatment and blood flow values were recorded for 10 minutes. The Moor VMS 1.0 software was used to analyze the recorded values and determine the average flow value and standard deviation of blood flow during the recording period. Blood flow experiments were also conducted 20 hours post treatment. The baseline value was determined by taking the average of averages for four untreated areas near the treatment site and comparing to the blood flow value measured at the previously treated site.

The effect of sodium nitrite concentration on nitric oxide production was determined by mixing 1 g samples of mask powder without sodium nitrite with 1 g of lyophilized crushed L. fermentum NCIMB 7230 microcapsules and hydrating with 10 mL of 5% glucose, 5% glucose with 5 mM sodium nitrite, 5% glucose with 10 mM sodium nitrite, 5% glucose with 20 mM sodium nitrite, or 5% glucose with 30 mM sodium nitrite. 1.5 g of each were then placed in 4 mL chambers and sealed. The head gas was sampled periodically and the nitric oxide concentration determined using a chemiluminescence NO analyzer (Sievers).

### Results:

Results of nitric oxide production by alginate skin mask containing L. fermentum NCIMB 7230 are shown in FIG. 21. An alginate skin mask was made composed of 5.5 g diatomaceous earth, 2 g sodium alginate, 2.2 g calcium sulphate dehydrate, 0.15 g sodium pyrophosphate and 0.12 g sodium nitrite (Mask). (1 and 2) This was hydrated with 6 ml distilled water and 100 μL aliquots were placed into sealed 2 mL vials. (3 and 4) 1 g of lyophilized L. fermentum NCIMB 7230 was added to 1 g of the mask mix and hydrated and sampled as above. The vials were incubated at room temperature and the head gas was sampled at 30-minute intervals. The nitric oxide content was measured with a chemiluminescence NO analyzer (Sievers).

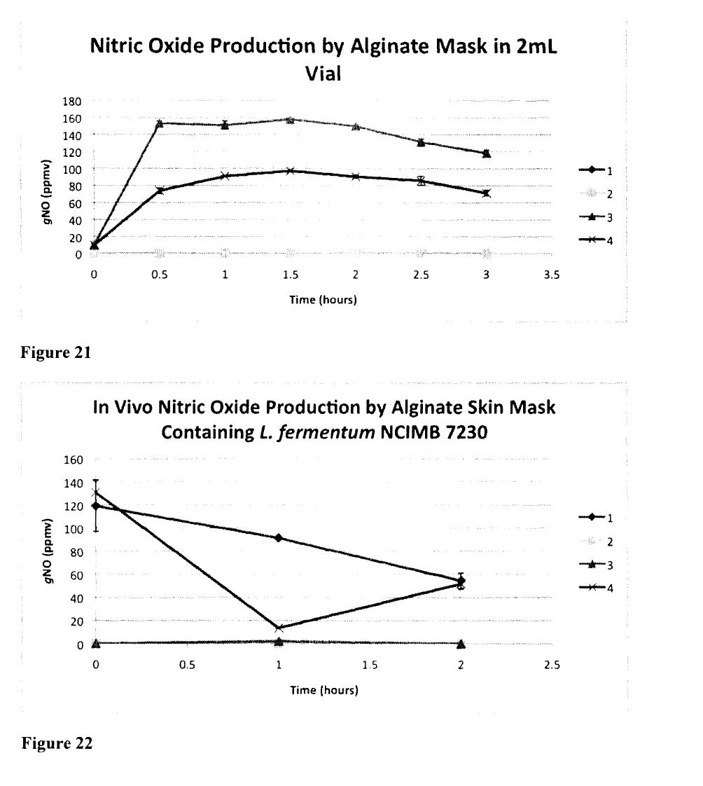

In vivo nitric oxide production by the alginate skin mask containing L. fermentum NCIMB 7230 is shown in FIG. 22. An alginate skin mask was made composed of 5.5 g diatomaceous earth, 2 g sodium alginate, 2.2 g calcium sulphate dehydrate, 0.15 g sodium pyrophosphate and 0.12 g sodium nitrite (Mask). This was hydrated with 6 ml distilled water and 100 μL aliquots were applied to the forearm of volunteers, surrounded by a spacer and covered with gas impermeable tape. (2) subject 1 control mask; (3) subject 2 control mask. 1 g of lyophilized L. fermentum NCIMB 7230 was added to 1 g of the mask mix and hydrated and sampled as above. (1) subject 1 active mask; (4) subject 2 active mask. The head gas was sampled at 30-minute intervals. The nitric oxide content was measured with a chemiluminescence NO analyzer (Sievers). The severe drop seen at the one-hour time point for subject 2 is attributed to a disruption in the seal on the testing chamber that was noted at the time of sampling.

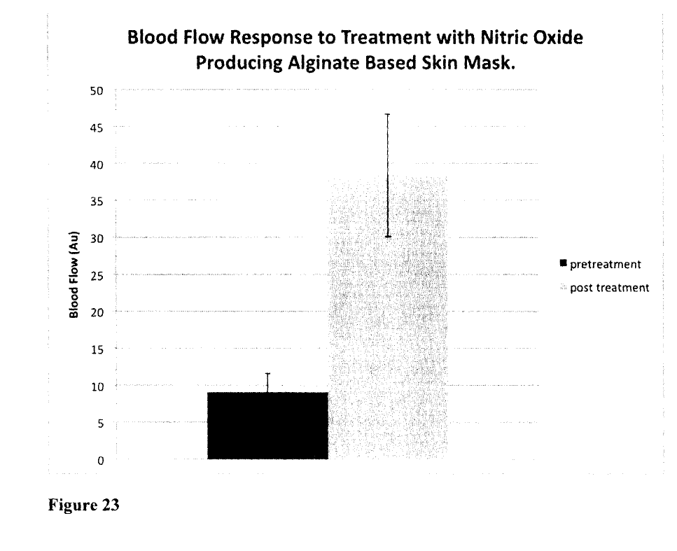

The blood flow response to treatment with nitric oxide producing alginate based skin mask is shown in FIG. 23. Using Blood flow changes were measured using a MoorVMS-LDF1 laser Doppler blood flow meter. An area was marked and a three minute baseline blood flow value was recorded. The marked area and surrounding skin was then covered with a mask containing lyophilized L. fermentum NCIMB 7230 hydrated with 6 volumes of water for three minutes. Then the marked area was exposed and the probe replaced while the surrounding area was still under treatment and blood flow values were recorded for 10 minutes. A 422% increase in blood flow was seen.

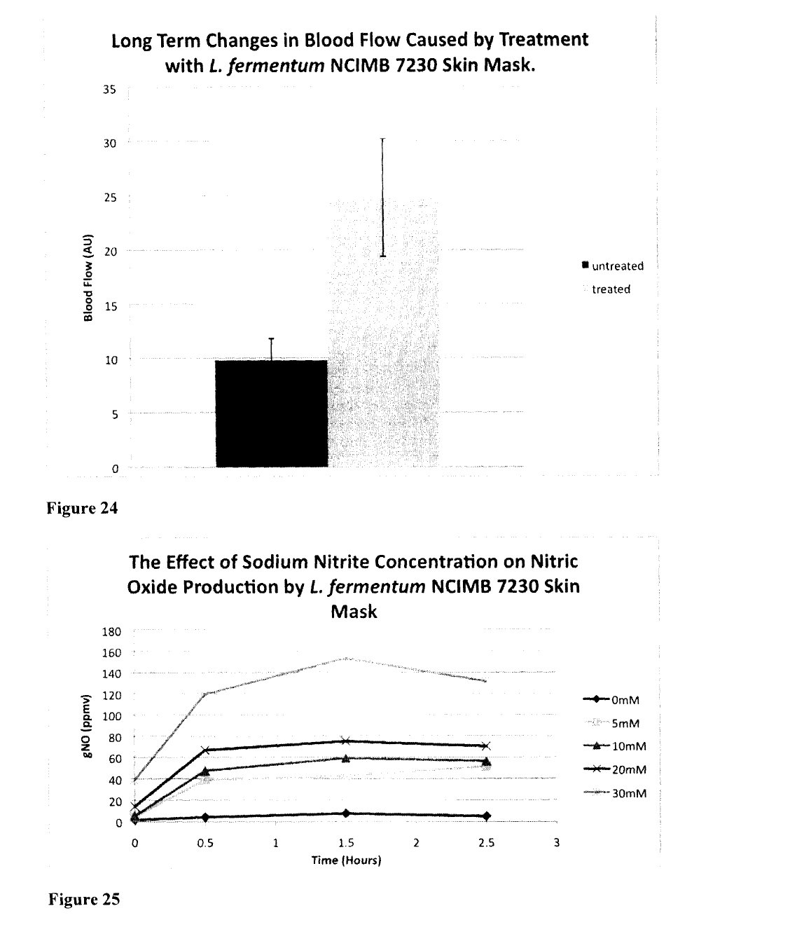

Long term changes in blood flow caused by treatment with the L. fermentum NCIMB 7230 skin mask are shown in FIG. 24. Blood flow at the treated site was measured with a MoorVMS-LDF1 laser Doppler blood flow meter 20 hours following a two hour treatment with L. fermentum NCIMB 7230 skin mask. The baseline value was determined by taking the average of blood flow averages for four untreated areas near the treatment site and comparing to the blood flow value measured at the previously treated site. The blood flow at the site of treatment was 253% higher than in surrounding untreated areas.

The effect of sodium nitrite concentration on nitric oxide production by the L. fermentum NCIMB 7230 nitric oxide face mask is shown in FIG. 25. 1 g samples of mask powder without sodium nitrite were mixed with 1 g of lyophilized, crushed L. fermentum NCIMB 7230 microcapsules and hydrating with 10 mL of 5% glucose, 5% glucose with 5 mM sodium nitrite, 5% glucose with 10 mM sodium nitrite, 5% glucose with 20 mM sodium nitrite, or 5% glucose with 30 mM sodium nitrite. 1.5 g of each were then placed in 4 mL chambers and sealed. The head gas was sampled periodically and the nitric oxide concentration determined using a chemiluminescence NO analyzer (Sievers).

### Bromelain Formulations

### Materials and Methods.

Solution preparation: Bromelain solutions were prepared by dissolving the lyophilised pineapple extract in water or in aqueous 8.5% sodium chloride. The pH of the resulting solution was adjusted to 7.0 with sodium hydroxide. Gelatin was weighed and added to the bromelain solution to the desired concentration. The concentration of sodium nitrite was set at 30 mM by addition of the right amount of a 1M stock solution.

Patch preparation: A one-sided gas permeable pocket was created by heat sealing 3 sides of a rectangular gas permeable membrane (Tegaderm) with a heat sealable plastic film. The resulting pocket was filled up with 10 mL of a bromelain solution and the fourth side of the pocket was heat sealed. A layer of aluminized tape was applied to the plastic film to avoid loss of gas.

NO measurements: Assay bottles were filled with 20 mL of the bromelain solutions and hermetically closed with a septum cap. Alternatively, patches were adhered to the top surface of an assay chamber so that the gas permeable membrane is exposed to the 5 mL chamber cavity. The bottle head gas, or the chamber gas was sampled through a septum and NO was measured with a chemiluminescence analyzer (Sievers).

### Results:

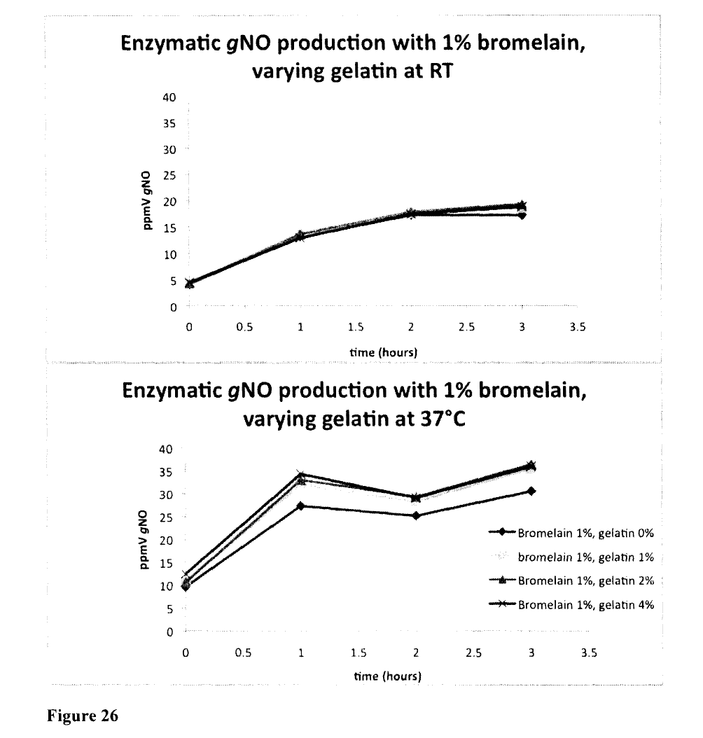

FIG. 26 shows the determination of gNO (ppmV) at Room Temperature (upper panel) and at 37° C. (lower panel) in the head gas of solutions containing 1 g Bromelain per 100 mL of aqueous saline (pH 7.0), 30 mM sodium nitrite, and varying concentrations of gelatin up to 4%. Measurements were performed by a chemiluminescence NO analyzer (Sievers).

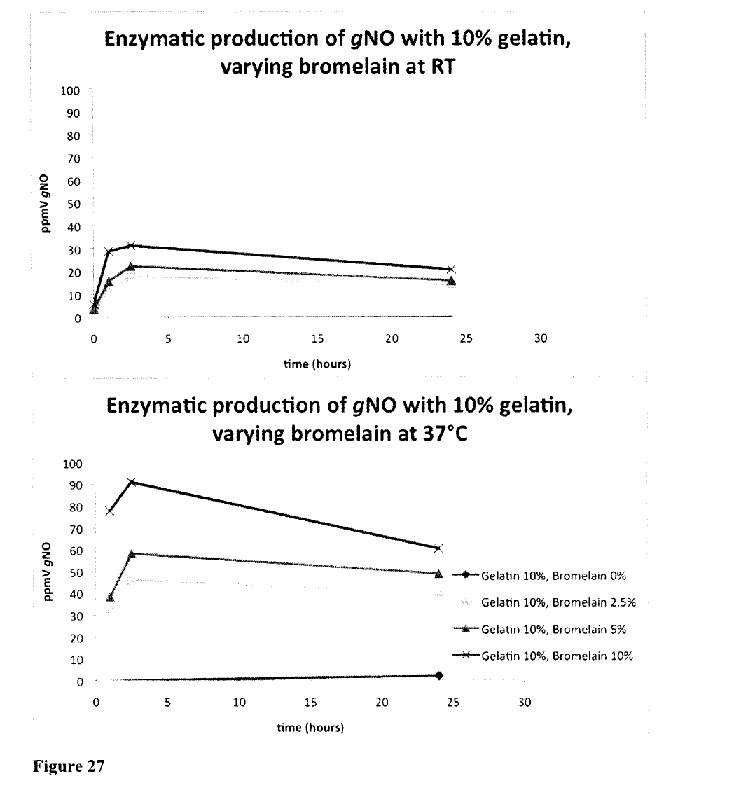

FIG. 27 shows the production of gNO (ppmV) measured at Room Temperature (upper panel) and at 37° C. (lower panel) on the head gas of solutions containing 8.5% sodium chloride, 10% gelatin, and varying concentrations of bromelain up to 10% (pH 7.0) in the presence of 30 mM sodium nitrite. Measurements were performed by a chemiluminescence NO analyzer (Sievers).

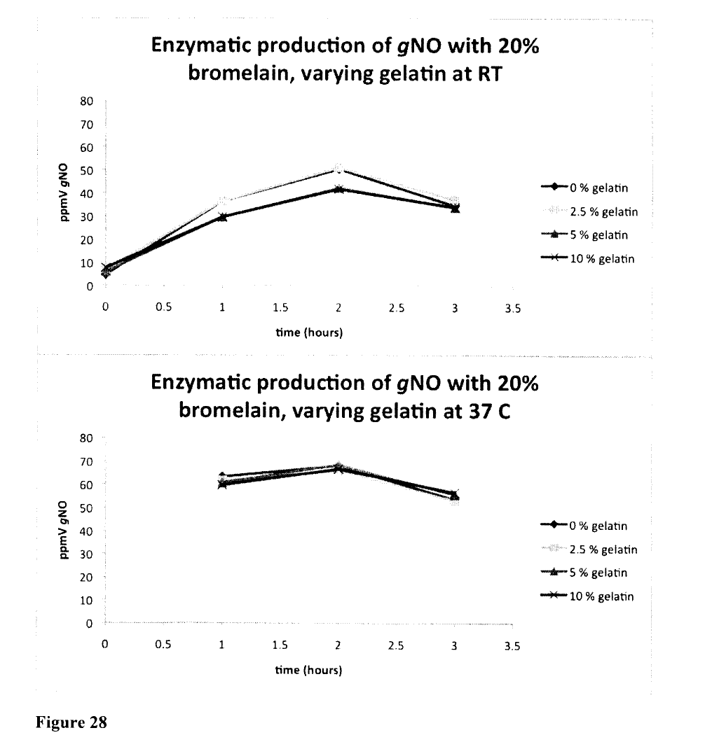

FIG. 28 shows the concentration of gNO (ppmV) in the head gas of solutions containing 10 g of bromelain per 100 mL of saline (pH 7.0), and varying concentrations of gelatin up to 10% in the presence of 30 mM sodium nitrite was determined at Room Temperature (upper panel) and at 37° C. (lower panel). Measurements were performed by a chemiluminescence NO analyzer (Sievers).

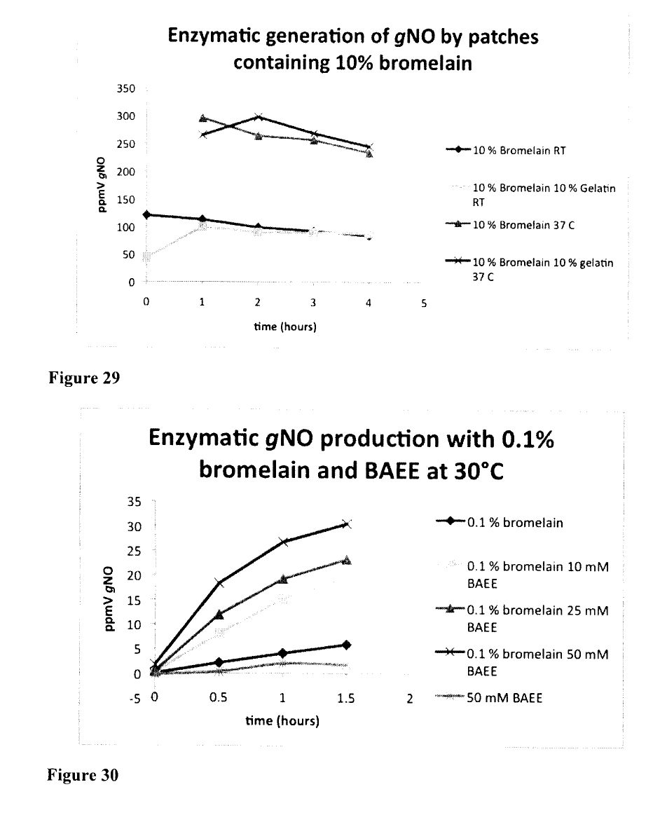

FIG. 29 shows the concentration of gNO produced by patches measured in 5 ml chambers at Room Temperature and at 37° C. The patches contained 10 g of bromelain per 100 mL of water (pH 7.0), 30 mM sodium nitrite, and 10% gelatin or no gelatin. Measurements were performed by a chemiluminescence NO analyzer (Sievers).

The enzymatic production of gNO by 0.1% bromelain and varying concentrations of its substrate BAEE was monitored every 30 minutes at 30° C. in the presence of 30 mM sodium nitrite and results are shown in FIG. 30. In the absence of enzyme (50 mM BAEE), the production of gNO was slightly above baseline. Stock solutions of 1% bromelain solution (pH 7.0) and 0.1 M N-A-Benzoyl L-arginine ethyl ester (BAEE)(pH 7.0) were mixed to obtain 0.1% bromelain solutions with varying concentrations of BAEE. Sodium nitrite was set at a final concentration of 30 mM. Production of gNO was monitored with a chemiluminescence analyzer (Sievers).

### Generation of gNO Using Enzyme (Esters, Esterases, or Lipases) and NaNO₃

The hydrolysis of either esters or triglycerides results in the production of acids and alcohol. Herein, it is proposed the use of the hydrolysis of esters to generate acid sustainably for up to 48 hours in order to catalyze the dismutation of an NO donor, optionally nitrite, and release at least 200 ppmV of gNO during the indicated period of time. Among the enzymes that catalyze the hydrolysis of esters, there is a distinction between esterases and lipases depending on the substrate preferences. Whereas esterases have higher affinities for esters of low molecular weight, lipases recognize mainly triglycerides of fatty acids although the specificity of each enzyme may vary considerably.

### Materials and Methods

Enzymatic Generation of gNO: A 200 μl reaction solution was prepared by combining water, an acetate ester (ethyl acetate, isobutyl acetate, octyl acetate) or a triglyceride such as triacetin (glyceryl triacetate), sodium nitrite, and an esterase (porcine liver esterase, rhyzopus oryzae esterase) or a lipase (porcine pancreatic lipase, candida rugosa lipase). The solution was then added to a 2 ml vial, which was closed tightly with a septum cap. The head gas was sampled every hour from the reaction containing vials in order to determine gNO concentrations.

Patch Preparation: A one-sided gas permeable pocket was created by heat-sealing 3 sides of a rectangular gas permeable membrane (Tegaderm) with a heat sealable plastic film. The resulting pocket was filled up with a triacetin/candida rugosa lipase/NaNO₂ solution and the fourth side of the pocket was then heat-sealed. A layer of aluminized tape was applied to the plastic film to avoid loss of gas. Lyophilised alginate microbeads were added to the solution in some patches to improve the consistency or physical properties of the device.

gNO Measurements: A known volume of gas was sampled hourly from the gas port of the assay chamber with a Hamilton syringe and gNO content was measured with a chemiluminescence analyzer (Sievers).

### Results and Discussion

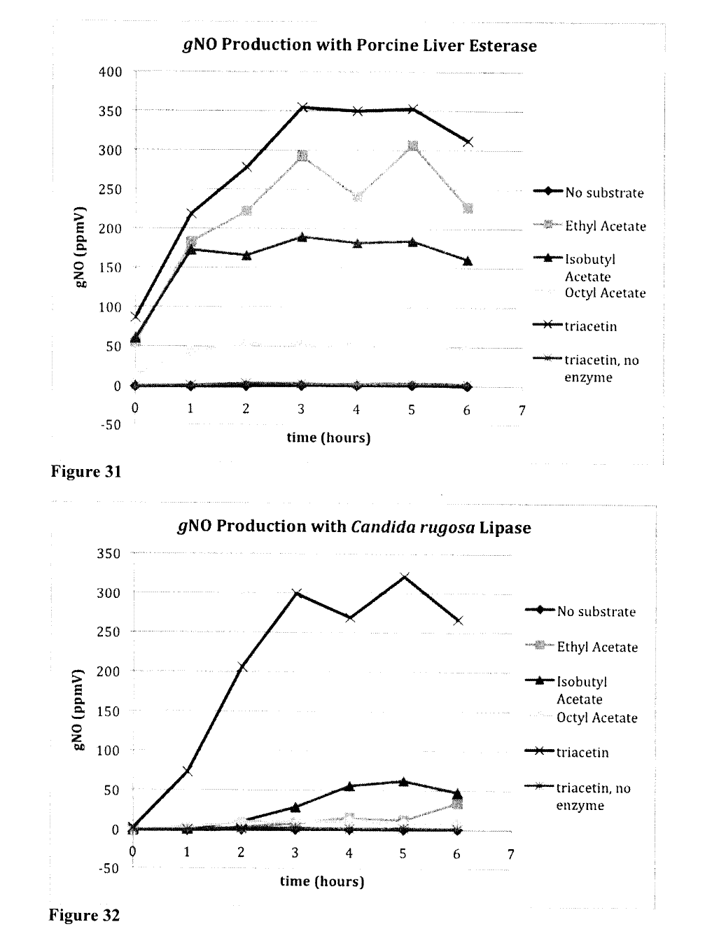

A number of enzymes are available for the hydrolysis of ester bonds. The advantage of utilizing the hydrolysis of esters or triglycerides is the reaction results in relatively innocuous by-products and weak acids. Using the right enzyme, with the right substrate, allows for the production of a nitric oxide producing dressing with minimal risk of toxicity. Work was performed to determine which enzymes could be used as well as the best possible substrate. FIG. 31 presents the results of experiments using porcine liver esterase against 4 substrates: Ethyl acetate, Isobutyl acetate, octyl acetate and triacetin. All 4 substrates produce acid upon hydrolysis by the enzyme, leading to nitric oxide production. Three of the substrates led to biologically relevant production of nitric oxide, reaching 200 ppmV in 1 hour. Triacetin was the strongest acid producer after hydrolysis, leading to upward of 350 ppmV over the 6 hour experiment.

Candida rugosa lipase is another enzyme able to hydrolyse ester bonds, though limited to triglyceride substrates. The enzyme was tested against four substrates and it was found that only triacetin, a simple triglyceride, was able to produce high amounts of nitric oxide (FIG. 32). The hydrolysis of triacetin by esterase or lipase leads to the production of glycerol and acetic acid, both innocuous compounds acceptable in cosmesis.

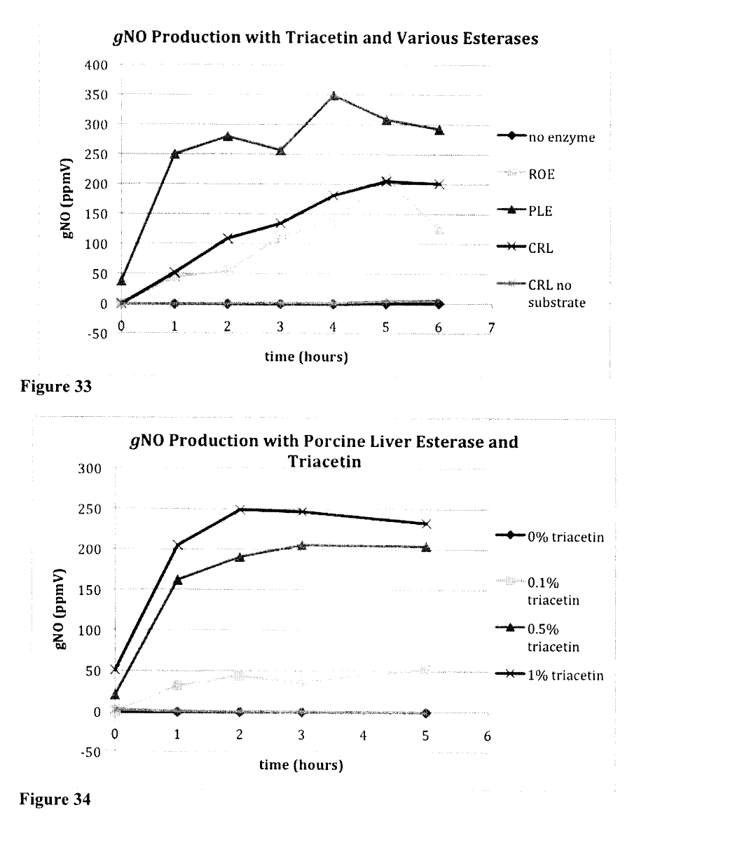

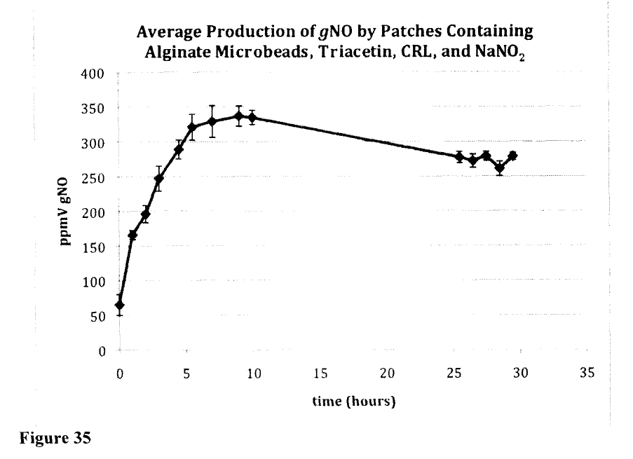

FIG. 33 presents an experiment testing three different esterase or lipase against triacetin. The comparison shows that porcine liver esterase reaches above 200 ppmV within an hour while the lipases take slightly more time. Both candida rugosa lipase and rhyzopus oryzae esterase also reach 200 ppmV but in 4-5 hours. It is important to note however that the concentration of enzyme will affect the time required to reach the maximum production of nitric oxide as well as the duration of production. Another element altering the level of nitric oxide produced by the enzymes is the substrate concentration of the assay. Varying the concentration of triacetin controls the production of nitric oxide (FIG. 34). The production can reach up to 250 ppmV using 1% triacetin in the assay while the use of 0.5% will limit the production to 200 ppmV. This interplay between enzyme and substrate allows for a fine adjustment of the level of production, an important aspect for cosmesis.

The enzymatic production of gNO was tested in dressings composed of Tegaderm (3M) non-occlusive dressings, polyethylene membrane and a gas impermeable upper layer of aluminium adhesive. The dressings were based on the use of candida rugosa lipase as the esterase and triacetin as the triglyceride substrate. FIG. 35 shows that production of nitric oxide rapidly reached the goal of 200 ppmV and was maintained at a biologically active level above 200 ppmV for 30 hours. This formulation can be used for cosmesis.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### SEQUENCE LIST

```
LOCUS YP_001271831, 375 aa, linear, BCT 6 Dec. 2007
DEFINITION Nitric-oxide synthase [Lactobacillus reuteri F275].
SOURCE Lactobacillus reuteri F275
ORIGIN
SEQ. ID. NO. 1
      1 mteqeqqtee lrcigcgsii qtedpnglgy tpksalekgk etgelycqrc
          frlrhyneia
    61 pvsltdddfl rllnqirdan alivyvvdvf dfngslipgl hrfvgdnpvl
          lvgnkedllp
  121 rslrrpkltd wirqqaniag lrpidtvlvs akknhqidhl ldviekyrhn
          rdvyvvgvtn
  181 vgkstlinqi ikqrtgvkel ittsrfpgtt ldkieipldd ghvlvdtpgi
          ihqeqmahvl
  241 spkdlkivap qkeikpktyq lndgqtlflg gvarfdylhg eragmvayfd
          nnlpihrtkl
  301 nnadnfyakh lgdlltppts deknefpple ryefhiteks divfeglgwi
          tvpakttvaa
  361 wvpkgvgalv rrami
LOCUS ZP_01273963, 1221 aa, linear, BCT 14 Apr. 2006
DEFINITION Nitrate reductase, alpha subunit [Lactobacillus reuteri
100-23].
SOURCE Lactobacillus reuteri 100-23
ORIGIN
SEQ. ID. NO. 2
      1 mksrffnkvd kfngtftqle ensrrwekly rqrwandkvv rtthgvnctg
          scswnvyvkq
    61 giitwehqat dypscgpnip gyeprgcprg asfswyeysp vrikypyirg
          klwelwtaak
  121 kehenpldaw asivedpeks kkykkvrghg glirvhryea lemisaacly
          tikkygpdri
  181 ggftpipams mmsfsagarf ialmggeqms fydwyadlpp aspqvwgeqt
          dvpesaewyn
  241 ssyiimwgsn vpltrtpdah fmtevrykgt kivayspdya envkfaddwl
          apepgsdsav
  301 aqamtyvild efyqkhpvkr fidyskrftd lpfmveleps tanddhytpg
          rfvrisdlvd
  361 ddtivnpawk tvvydqnnhk ivvpngtmgq eynvkekwnl elldqngnki
          dpalsindqg
  421 geteqiiadf pafsndgnsv vqrhlpvkkl kftdgqehlv tnvydlmmaq
          mgidrtgndd
  481 laakdamdae syftpawqes rsgvkaeqvi qiarefaqna aenegrsmvi
          mgggvnhwfn
  541 admnyrniin mlmlcgcvgm tgggwahyvg qeklrpqegw anitfandwe
          kggarqmqgt
  601 twyyfatdqw ryeeidnqaq kspvwkskhs ylhnadynqm airlgwlpsy
          pqfdrnplsf
  661 akdynttdid eiskkvvdel kkgtlhfaae dpdanqnqpk afflwrsnlf
          assgkgaeyf
  721 mkhllgaeng llakpndrvk pqdmiwrdkg avgkldlvvd mdfrmvstpm
          ysdvvlpaat
  781 wyekkdlsst dmhpfihpfn aaispmwesk sdwqqfklla ktisemakky
          mpgtfydlks
  841 aplghntqge iaqpygkikd wkngetepip gktmpslklv trdytkiydk
          fitlgpnivn
  901 nygykvddqy dylkgmngta segigagcpl ldedekvcda ilrmstasng
          kladrawekk
1961 qertgehltd igrghaddsm sfkqitaqpq eayptpigts akhggarytp
          fslmternip
1021 tftltgrqhf yidheifref genmatykps lppvvmapgd vdvppvkdev
          tlkymtphgk
1081 wnihtmyydn lemltlfrgg ptiwispqda dkikvkdndw ievynrngvv
          taravvsvrm
1141 pegsmymyha qdneiyepls titgnrggsh naptqihvkp thmvggygql
          sygwnyygpt
1201 gnqrdlyanv rklrkvnwse d
LOCUS ZP_01273962, 519 aa, linear, BCT 14 Apr. 2006
DEFINITION Nitrate reductase, beta subunit [Lactobacillus reuteri
100-23].
SOURCE Lactobacillus reuteri 100-23
ORIGIN
SEQ. ID. NO. 3
      1 mkikaqismv lnldkcigch tcsvtckntw tnrpgaeymw fnnvetkpgv
          gypkrweded
    61 qyhggwtlns kgklklrags klnkialgki fynndmpeld nyyepwtydy
          ktlfgpeqkh
  121 qpvarpksqi tgegmelttg pnwdddlags teyvqqdpnm qkiegdiknn
          feqafmmylp
  181 rlcehclnap cvascpsgam ykrdedgivl vdqercrgwr fcmtgcpykk
          vyfnwkthka
  241 ekctfcypri eegptvcae tcvgriryig ailydadrve eaastpdesk
          lyeaqlglfl
  301 dpndpevvkq alkdgiseem ieaaqkspiy kmavkekiaf plhpeyrtmp
          mvwyipplsp
  361 vmsyfegrds iknpemifpg idqmrvpvqy laslltagnv pvikkalykl
          ammrlymrak
  421 tsgrdfdssk lervdlteer atslyrllai akyedrfvip ssqkaemeda
          qteggslgyd
  481 ecegcalapq hksmfkkaea gkstnqiyad sfyggiwrd
LOCUS ZP_01273960, 229 aa, linear, BCT 14 Apr. 2006
DEFINITION Nitrate reductase, gamma subunit [Lactobacillus reuteri
100-23].
SOURCE Lactobacillus reuteri 100-23
ORIGIN
SEQ. ID. NO. 4
      1 mhngwsiflw viypyimlas ffigtfvrfk yfhpsitaks selfekkwlm
          igsitfhigi
    61 ilaffghclg mfipaswtay fgitehmyhi fgslmmgipa gilafvgiai
          ltyrrmtcsr
  121 vyktsdindi ivdwallvti alglactitg afidynyrtt ispwarslfv
          lnpqwqlmrs
  181 vpliykihvl cglaifgyfp ytrlvhaltl pwqyifrrfi vyrrrarvy
LOCUS ZP_01273961, 192 aa, linear, BCT 14 Apr. 2006
DEFINITION Nitrate reductase, delta subunit [Lactobacillus reuteri
100-23].
SOURCE Lactobacillus reuteri 100-23
ORIGIN
SEQ. ID. NO. 5
      1 midfrrltdl kdtfavlsrl idypdtetfs peirqllltd nalstatrge
          llslfdelaa
    61 lssievqemy ahlfemnrry tlymsyykmt dsrergtila rlkmlyemfg
          iseatselsd
  121 ylplllefla ygdytndprr qdiglalsvi edgtytllkn avtdsdnpyi
          qlirltrsii
  181 gscikmevre da
```

TABLE 2
Nitric oxide (NO) biosynthesis from arginine by nitric oxide
synthase (NOS) in the presence of oxygen and NADPH

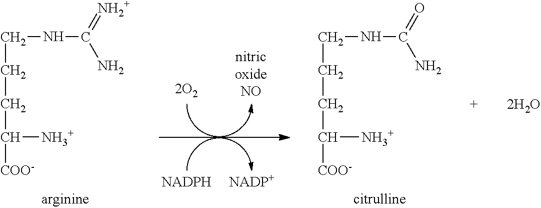

TABLE 3
Nitric oxide (NO) production by reduction of nitrite (NO₂) salts
NO₂ + 2H+ → H₂O + NO

TABLE 4
nitric oxide (NO) production by reduction of
nitrate (NO₃) salts to nitrite (NO₂) and then
reduction of NO₂ to nitric oxide gas gNO

| | |
|---|---|
| (Reaction 1) | 2NO₃ [symbol] 2NO₂ + O₂ |
| (Reaction 2) | NO₂ + 2H⁺ → H₂O + NO |

### REFERENCES

Archer, S. Measurement of Nitric Oxide in Biological Systems. The FASEB Journal 7, 349-360 (1993).
Benjamin, N., Pattullo, S., Weller, R., Smith, L., & Ormerod, A. Wound licking and nitric oxide. Lancet 349, 1776 (1997).
Hardwick, J. B., Tucker, A. T., Wilks, M., Johnston, A., & Benjamin, N. A novel method for the delivery of nitric oxide therapy to the skin of human subjects using a semi-permeable membrane. Clin. Sci. (Lond) 100, 395-400 (2001).
Kikuchi, K., Nagano, T. and Hirobe, M. Novel detection method of nitric oxide using horseradish peroxidase. Biol. Pharm. Bull. 1996 April; 19(4):649-651.
Liu, Y. & Wang, M. W. Botanical drugs: challenges and opportunities: contribution to Linnaeus Memorial Symposium 2007. Life Sci. 82, 445-449 (2008).
Michelakis, E. D. & Archer, S. L. The measurement of NO in biological systems using chemiluminescence. Methods Mol. Biol. 100, 111-127 (1998).
Serarslan, G., Altug, E., Kontas, T., Atik, E., & Avci, G. Caffeic acid phenethyl ester accelerates cutaneous wound healing in a rat model and decreases oxidative stress. Clin. Exp. Dermatol. 32, 709-715 (2007).
Syed, D. N., Afaq, F., & Mukhtar, H. Pomegranate derived products for cancer chemoprevention. Semin. Cancer Biol. 17, 377-385 (2007).
Vayalil, P. K., Elmets, C. A., & Katiyar, S. K. Treatment of green tea polyphenols in hydrophilic cream prevents UVB-induced oxidation of lipids and proteins, depletion of antioxidant enzymes and phosphorylation of MAPK proteins in SKH-1 hairless mouse skin. Carcinogenesis 24, 927-936 (2003).
Weller, R. et al. Nitric oxide is generated on the skin surface by reduction of sweat nitrate. J. Invest Dermatol. 107, 327-331 (1996).
Wolf, G., Arendt, E. K., Pfahler, U., & Hammes, W. P. Heme-dependent and heme-independent nitrite reduction by lactic acid bacteria results in different N-containing products. Int. J. Food Microbiol. 10, 323-329 (1990).
Xu, J. & Verstraete, W. Evaluation of nitric oxide production by lactobacilli. Appl. Microbiol. Biotechnol. 56, 504-507 (2001).

1. (canceled)

2. A method for skin cosmesis in a subject in need thereof comprising:
(a) contacting the skin with (i) nitric oxide gas releasing composition, the composition containing a plurality of inactive agents that, upon activation, react to produce nitric oxide gas or (ii) a device comprising a casing permeable to nitric oxide gas, the casing containing a plurality of inactive agents that, when activated, react to produce nitric oxide gas;
(b) activating the inactive agents to produce nitric oxide gas,
wherein the nitric oxide gas contacts the skin for cosmesis in the subject in need thereof; and wherein the inactive agents comprise i) a nitric oxide gas precursor, ii) (a) an isolated enzyme or a live cell expressing an endogenous enzyme, the enzyme having activity that converts the nitric oxide gas precursor to nitric oxide gas or having activity on a substrate that produces a catalyst that causes the conversion of the nitric oxide gas precursor to nitric oxide gas or (b) a live cell producting a catalyst for converting nitric oxide gas precursor to nitric oxide gas; and a carrier.

3. (canceled)

4. The method of claim 1, wherein the inactive agents comprise separated agents and activating the separated agents comprises combining the separated agents.

5. The method of claim 4, wherein the separated agents are activated in step (b) by mixing the separated agents by applying pressure or temperature to the device.

6. The method of claim 1, wherein the inactive agents are dehydrated agents and activating the inactive agents comprises hydrating the agents.

7. A method for skin cosmesis in a subject in need thereof comprising:
contacting the skin with a nitric oxide gas releasing composition or a device comprising the nitric oxide gas releasing composition, the composition comprising an isolated enzyme or a live cell expressing an endogenous enzyme, the enzyme (i) having activity that converts nitric oxide gas precursor to nitric oxide gas or (ii) having activity on a substrate that produces a catalyst that causes the conversion of the nitric oxide gas precursor to nitric oxide gas or (b) a live cell expressing a catalyst for converting the nitric oxide gas precursor to nitric oxide gas; and a carrier;
wherein the composition reacts with nitrate in sweat on the skin to produce nitric oxide gas for cosmesis in the subject in need thereof or wherein the composition further comprises the nitric oxide gas precursor to produce nitric oxide gas for cosmesis in the subject in need thereof.

8.-11. (canceled)

12. The method of claim 7, wherein the carrier comprises a matrix selected from a natural polymer, a synthetic polymer, a hydrogel, a natural gel, dissolvable film, multi-part or layered dissolvable film, a microcapsule, a liposome, hydrocarbon-based and petroleum jelly.

13.-18. (canceled)

19. The method of claim 7, wherein the composition is a cream, slab, gel, hydrogel, dissolvable film, spray, paste, emulsion, patch, liposome, balm or mask.

20. The method of claim 7, wherein the device for delivering nitric oxide gas to skin comprises a casing comprising a barrier surface and a contact surface, said contact surface being permeable to nitric oxide gas, wherein the casing contains the composition, the composition located between the barrier surface and the contact surface.

21. (canceled)

22. The method of claim 20, wherein the casing separates the composition from the tissue and the casing is impermeable to the composition.

23.-27. (canceled)

28. The method of claim 7, wherein the enzyme is a nitrate reductase (NaR), nitrite reductase (NiR), nitric oxide synthase (NOS), glutathione S-transferase (GST), or cytochrome P450 system (P450).

29. The method of claim 7, wherein the catalyst comprises protons and wherein the protons are a product or by-product of the enzyme reaction.

30. (canceled)

31. (canceled)

32. The method of claim 7, wherein the enzyme and substrate comprise lipase and triglyceride, esterase and ester, lipase and ester, esterase and triglyceride, protease and protein, trypsin and protein, chymotrypsin and protein.

33. The method of claim 7 wherein the enzyme is lipase and the substrate is triacetin.

34. (canceled)

35. (canceled)

36. The method of claim 7, wherein a reducing agent and/or an enzyme cofactor is added.

37. (canceled)

38. The method of claim 20, wherein the barrier surface is impermeable to oxygen.

39.-45. (canceled)

46. The method of claim 7, wherein the cell is a probiotic microorganism of the genus Lactobacillus, Bifidobacteria, Pediococcus, Streptococcus, Enterococcus, or Leuconostoc.

47.-51. (canceled)

52. The method of claim 7, wherein the cell is microencapsulated.

53.-55. (canceled)

56. The method of claim 7, wherein the cell or enzyme or precursor is immobilized in a reservoir.

57. (canceled)

58. (canceled)

59. The method of claim 7, wherein the composition further comprises growth media for cells such as MRS broth, LB broth, glucose, or other carbon source containing growth media.

60. (canceled)

61. The method of claim 7, wherein i) the enzyme comprises nitrite reductase (NiR) and the nitric oxide gas precursor comprises a nitrite or salt thereof, ii) the enzyme comprises nitric oxide synthase (NOS) and the nitric oxide precursor comprises L-arginine, or iii) the enzyme comprises nitrate reductase (NaR) and the nitric oxide gas precursor is a nitrate or salt thereof.

62.-70. (canceled)

71. The method of claim 20, wherein the device further comprises a nitric oxide gas concentrating substance comprising a spacer, a gas cell containing structure, or a sponge for collection of the nitric oxide gas.

72.-75. (canceled)

76. The method of claim 20, wherein the casing comprises a plurality of layers, wherein the layers comprise:
a) a barrier layer;
b) a contact layer; and
c) an active layer.

77. (canceled)

78. The method of claim 76, further comprising a reservoir layer.

79. (canceled)

80. (canceled)

81. The method of claim 78, further comprising at least one valve connecting the active layer and the reservoir layer, wherein the valve has an initial closed position in which the cell or enzyme are separate from the precursor and an open position in which the active layer and reservoir layer are in fluid communication, and the cell or enzyme or precursor are permitted to flow between the layers.

82. The method of claim 81, wherein the valve comprises a one-way valve, and wherein in the open position either the enzyme or cell or the precursor is permitted to flow between the layers or wherein the valve comprises a pressure actuated valve that is actuable from the closed position to the open position by compression of the device.

83.-85. (canceled)

86. The method of claim 78, wherein the nitric oxide (NO) is produced in a chemical reaction between an acid produced by a lactic acid producing bacteria (LAB) in the active layer and an NO containing substrate in the reservoir layer.

87. The method of claim 7, wherein the composition has an inactive composition state and an active composition state, wherein in the inactive composition state, the composition is dehydrated and the precursor does not interact with the enzyme or catalyst to produce NO gas and wherein in the active composition state, the composition is hydrated and the precursor is converted to NO gas by the enzyme or catalyst.

88.-90. (canceled)

91. The method of claim 7, wherein the subject is a human.


SEQUENCE LISTING (5 sequences)

SEQ ID NO: 1 - protein, 375 aa, Lactobacillus reuteri F275
MTEQEQQTEELRCIGCGSIIQTEDPNGLGYTPKSALEKGKETGELYCQRCFRLRHYNEIAPVSLTDDDFLRLLNQIRDANALIVYVVDVFDFNGSLIPGLHRFVGDNPVLLVGNKEDLLPRSLRRPKLTDWIRQQANIAGLRPIDTVLVSAKKNHQIDHLLDVIEKYRHNRDVYVVGVTNVGKSTLINQIIKQRTGVKELITTSRFPGTTLDKIEIPLDDGHVLVDTPGIIHQEQMAHVLSPKDLKIVAPQKEIKPKTYQLNDGQTLFLGGVARFDYLHGERAGMVAYFDNNLPIHRTKLNNADNFYAKHLGDLLTPPTSDEKNEFPPLERYEFHITEKSDIVFEGLGWITVPAKTTVAAWVPKGVGALVRRAMI

SEQ ID NO: 2 - protein, 1221 aa, Lactobacillus reuteri 100-23
MKSRFFNKVDKFNGTFTQLEENSRRWEKLYRQRWAHDKVVRTTHGVNCTGSCSWNVYVKQGIITWEHQATDYPSCGPNIPGYEPRGCPRGASFSWYEYSPVRIKYPYIRGKLWELWTAAKKEHENPLDAWASIVEDPEKSKKYKKVRGHGGLIRVHRYEALEMISAACLYTIKKYGPDRIGGFTPIPAMSMMSFSAGARFIALMGGEQMSFYDWYADLPPASPQVWGEQTDVPESAEWYNSSYIIMWGSNVPLTRTPDAHFMTEVRYKGTKIVAVSPDYAENVKFADDWLAPEPGSDSAVAQAMTYVILDEFYQKHPVKRFIDYSKRFTDLPFMVELEPSTANDDHYTPGRFVRISDLVDDDTIVNPAWKTVVYDQNNHKIVVPNGTMGQEYNVKEKWNLELLDQNGNKIDPALSINDQGGETEQIIADFPAFSNDGNSVVQRHLPVKKLKFTDGQEHLVTNVYDLMMAQMGIDRTGNDDLAAKDAMDAESYFTPAWQESRSGVKAEQVIQIAREFAQNAAENEGRSMVIMGGGVNHWFNADMNYRNIINMLMLCGCVGMTGGGWAHYVGQEKLRPQEGWANITFANDWEKGGARQMQGTTWYYFATDQWRYEEIDNQAQKSPVWKSKHSYLHNADYNQMAIRLGWLPSYPQFDRNPLSFAKDYNTTDIDEISKKVVDELKKGTLHFAAEDPDANQNQPKAFFLWRSNLFASSGKGAEYFMKHLLGAENGLLAKPNDRVKPQDMIWRDKGAVGKLDLVVDMDFRMVSTPMYSDVVLPAATWYEKKDLSSTDMHPFIHPFNAAISPMWESKSDWQQFKLLAKTISEMAKKYMPGTFYDLKSAPLGHNTQGEIAQPYGKIKDWKNGETEPIPGKTMPSLKLVTRDYTKIYDKFITLGPNIVNNYGYKVDDQYDYLKGMNGTASEGIGAGCPLLDEDEKVCDAILRMSTASNGKLADRAWEKKQERTGEHLTDIGRGHADDSMSFKQITAQPQEAYPTPIGTSAKHGGARYTPFSLMTERNIPTFTLTGRQHFYIDHEIFREFGENMATYKPSLPPVVMAPGDVDVPPVKDEVTLKYMTPHGKWNIHTMYYDNLEMLTLFRGGPTIWISPQDADKIKVKDNDWIEVYNRNGVVTARAVVSVRMPEGSMYMYHAQDNEIYEPLSTITGNRGGSHNAPTQIHVKPTHMVGGYGQLSYGWNYYGPTGNQRDLYANVRKLRKVNWSED

SEQ ID NO: 3 - protein, 519 aa, Lactobacillus reuteri 100-23
MKIKAQISMVLNLDKCIGCHTCSVTCKNTWTNRPGAEYMWFNNVETKPGVGYPKRWEDEDQYHGGWTLNSKGKLKLRAGSKLNKIALGKIFYNNDMPELDNYYEPWTYDYKTLFGPEQKHQPVARPKSQITGEGMELTTGPNWDDDLAGSTEYVQQDPNMQKIEGDIKNNFEQAFMMYLPRLCEHCLNAPCVASCPSGAMYKRDEDGIVLVDQERCRGWRFCMTGCPYKKVYFNWKTHKAEKCTFCYPRIEEGQPTVCAETCVGRIRYIGAILYDADRVEEAASTPDESKLYEAQLGLFLDPNDPEVVKQALKDGISEEMIEAAQKSPIYKMAVKEKIAFPLHPEYRTMPMVWYIPPLSPVMSYFEGRDSIKNPEMIFPGIDQMRVPVQYLASLLTAGNVPVIKKALYKLAMMRLYMRAKTSGRDFDSSKLERVDLTEERATSLYRLLAIAKYEDRFVIPSSQKAEMEDAQTEQGSLGYDECEGCALAPQHKSMFKKAEAGKSTNQIYADSFYGGIWRD

SEQ ID NO: 4 - protein, 229 aa, Lactobacillus reuteri 100-23
MHNGWSIFLWVIYPYIMLASFFIGTFVRFKYFHPSITAKSSELFEKKWLMIGSITFHIGIILAFFGHCLGMFIPASWTAYFGITEHMYHIFGSLMMGIPAGILAFVGIAILTYRRMTCSRVYKTSDINDIIVDWALLVTIALGLACTITGAFIDYNYRTTISPWARSLFVLNPQWQLMRSVPLIYKIHVLCGLAIFGYFPYTRLVHALTLPWQYIFRRFIVYRRRARVY

SEQ ID NO: 5 - protein, 192 aa, Lactobacillus reuteri 100-23
MIDFRRLTDLKDTFAVLSRLIDYPDTETFSPEIRQLLLTDNALSTATRGELLSLFDELAALSSIEVQEMYAHLFEMNRRYTLYMSYYKMTDSRERGTILARLKMLYEMFGISEATSELSDYLPLLLEFLAYGDYTNDPRRQDIQLALSVIEDGTYTLLKNAVTDSDNPYIQLIRLTRSLIGSCIKMEVREDA